(19) United States
(12) Patent Application Publication
(10) Pub. No.: US 2003/0040683 A1
(43) Pub. Date: Feb. 27, 2003

(54) Site selection for determining analyte concentration in living tissue

(76) Inventors: Peter Rule, Los Altos Hills, CA (US); James R. Braig, Piedmont, CA (US); Daniel S. Goldberger, Boulder, CO (US); Julian M. Cortella, Alameda, CA (US); Heidi M. Smith, Union City, CA (US); Roger O. Herrera, Emeryville, CA (US); Kenneth G. Witte, San Jose, CA (US); Philip C. Hartstein, Cupertino, CA (US); Mark D. Agostino, Alameda, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/189,978

(22) Filed: Jul. 3, 2002

Related U.S. Application Data
(60) Provisional application No. 60/303,475, filed on Jul. 6, 2001.
(60) Provisional application No. 60/339,246, filed on Nov. 12, 2001.
(60) Provisional application No. 60/338,992, filed on Nov. 13, 2001.
(60) Provisional application No. 60/336,294, filed on Oct. 29, 2001.

(51) Int. Cl. A61B 5/00

(52) U.S. Cl. 600/584

(57) ABSTRACT

A device and method for selecting and stabilizing proper sites for the measurement of the concentration of an analyte, for example glucose, within the tissue of a subject or patient are disclosed. One embodiment of the device immobilizes the subject's forearm and finger, thereby stabilizing measurement sites thereon for exposure to a noninvasive monitor which captures analyte concentration data within the subject's skin. The method involves the choice of a location on the subject's body at which to take the analyte measurement, preferably based on the amount of time that has elapsed since the last time the subject ate.

### RELATED APPLICATIONS

This application claims priority to U.S. Provisional Patent Application No. 60/303,475, filed Jul. 6, 2001; No. 60/339,246, filed Nov. 12, 2001; and No. 60/338,992, filed Nov. 13, 2001, all entitled SITE SELECTION FOR DETERMINING ANALYTE CONCENTRATION IN LIVING TISSUE, as well as U.S. Provisional Patent Application No. 60/336,294, filed Oct. 29, 2001 and entitled METHOD AND DEVICE FOR INCREASING ACCURACY OF BLOOD CONSTITUENT MEASUREMENT, the entire contents of all of which are hereby incorporated by reference herein and made a part of this specification.

### BACKGROUND OF THE INVENTION

1. Field of the Invention

This invention relates generally to determining analyte concentrations within living tissue. More particularly, this invention relates to a device and method for isolating and stabilizing regions of living tissue for consistent determination of analyte concentration therein.

2. Description of the Related Art

Millions of diabetics are forced to draw blood on a daily basis to determine their blood glucose levels. A search for a methodology to accurately determine blood glucose levels has been substantially expanded in order to alleviate the discomfort of these individuals.

### SUMMARY OF THE INVENTION

A significant advance in the state of the art of blood glucose analysis has been realized by an apparatus taught in Assignee's U.S. Pat. No. 6,198,949, entitled SOLID-STATE NONINVASIVE INFRARED ABSORPTION SPECTROMETER FOR THE GENERATION AND CAPTURE OF THERMAL GRADIENT SPECTRA FROM LIVING TISSUE, issued Mar. 6, 2001, and by methodology taught in Assignee's U.S. Pat. No. 6,161,028, entitled METHOD FOR DETERMINING ANALYTE CONCENTRATION USING PERIODIC TEMPERATURE MODULATION AND PHASE DETECTION, issued Dec. 12, 2000, as well as the methods and apparatus taught in Assignee's U.S. patent application Ser. No. 09/538,164, filed Mar. 30, 2000, entitled METHOD AND APPARATUS FOR DETERMINING ANALYTE CONCENTRATION USING PHASE AND MAGNITUDE DETECTION OF A RADIATION TRANSFER FUNCTION, and Ser. No. 09/427,178, filed Oct. 25, 1999, entitled SOLID-STATE NON-INVASIVE THERMAL CYCLING SPECTROMETER. The entire disclosure of each of the above-mentioned patents and patent applications is hereby incorporated by reference herein.

U.S. Pat. No. 6,198,949 discloses a spectrometer for noninvasive transfer of thermal gradient spectra to and from living tissue. The spectrometer includes an infrared transmissive thermal mass, referred to as a thermal mass window, for inducing a transient temperature gradient in the tissue by means of conductive heat transfer with the tissue, and a cooling system in operative combination with the thermal mass for the cooling thereof. Also provided is an infrared sensor for detecting infrared emissions from the tissue as the transient temperature gradient progresses into the tissue, and for providing output signals proportional to the detected infrared emissions. A data capture system is provided for sampling the output signals received from the infrared sensor as the transient temperature gradient progresses into to the tissue. The transient thermal gradients arising due to the intermittent heating and cooling of the subject's skin generate thermal spectra which yield very good measurements of the subject's blood glucose levels.

Although the apparatus taught in the above-mentioned U.S. Pat. No. 6,198,949 has led to a significant advance in the state of the art of blood glucose analysis, greater accuracy can still be achieved. If several separate measurements are required, it follows that the thermal mass window must be brought into contact with the subject's skin several times. The problem with this is that each of such contacts tends to be slightly different. For instance, slight differences in skin topology and/or pressure may arise at the interface between the thermal mass window and the skin; the subject may move that portion of his or her body, for instance the arm, which is in contact with the thermal mass window; and muscular tension may change between measurements. Each of these factors, and perhaps others as well, tend to complicate the already complex nature of the contact between the skin and the thermal mass window.

Thus, in one embodiment uniformity is achieved by employing a device and/or method for selecting and stabilizing proper sites for the measurement of the concentration of an analyte, for example glucose, within the tissue of a subject or patient. One embodiment of the device immobilizes the subject's forearm and finger, thereby stabilizing measurement sites thereon for exposure to a monitor which captures analyte concentration data within the subject's skin. The method involves the choice of a location on the subject's body at which to take the analyte measurement, preferably based on the amount of time that has elapsed since the last time the subject ate. The device and/or method can be applied to noninvasive as well as invasive measurement techniques.

A restricted period commences after the subject eats. This restricted period is characterized by a restriction on where the subject may take analyte measurements; specifically, the subject is restricted to taking measurements “on-site” (on a finger or fingertip, or alternatively, anywhere distal of the wrist) during the restricted period. In one embodiment, the restricted period lasts from about 0.5 to about 3 hours. In another embodiment, the restricted period lasts from about 1.0 to about 2 hours. In another embodiment, the restricted period lasts from about 1.5 to about 2 hours. In a presently preferred embodiment, the restricted period lasts about 2 hours.

In contrast, when no restricted period is in effect (i.e., the designated time interval has elapsed since the last time the subject ate) the subject may take analyte measurements either on-site or at an alternative site such as, for example, the forearm. It is to be understood, however, that “alternative site” refers to any location other than the on-site positions.

In one embodiment, the subject or operator takes analyte measurements by drawing a sample of blood from the measurement site and analyzing the blood with any of the various known and commercially available optical or electrochemical devices, test strips, etc. designed for analysis of blood samples drawn from the subject. In another embodiment, the subject takes analyte measurements with a noninvasive monitor, including for example a monitor of the type which detects infrared energy emitted and/or reflected by the subject's tissue to determine the analyte concentration based on the amount of infrared energy absorbed by the analyte. It is to be understood, however, that “noninvasive monitor” refers to any type of monitor which does not analyze a blood sample drawn from the subject. In another embodiment, the subject employs a mix of blood-drawing and noninvasive measurement techniques, for example using one of the techniques only during the restricted period and the other only when the restricted period is not in effect, and/or using one of the techniques only on-site and the other only for alternative-site measurements. As a further alternative the subject could use either technique at any time of the day and/or at either type of measurement location.

Advantageously, a mechanical stabilization device, such as a finger/elbow brace, tube, slot, etc. could be employed to immobilize the subject's finger and/or hand when exposing it to a monitor, such as an invasive or noninvasive monitor for on-site measurements. Thus, in one embodiment an apparatus is adapted to take on-site analyte measurements. The apparatus is adapted to stabilize the subject's finger and/or hand with respect to the apparatus so that accurate measurements can be made. In another embodiment the device is adapted to stabilize both on-site and alternative-site locations.

In another embodiment, excitation of the dermis is used to induce increased blood flow in preparation for a measurement of a blood constituent, such as glucose and/or alcohol, at the excited location. The measurement may preferably be performed noninvasively, or by any other known technique. Excitation can be achieved by applying heat to the location in question. Alternatively, excitation is achieved via local application of a vacuum or by rubbing the skin to increase circulation. As further alternatives, excitation can be achieved via topical application of an irritant or vasodilating substance to the area which will be tested. A pharmacological agent, chemical, drug, or other substance or method that will cause systemic vasodilation in the entire body or to a specific region, can also be employed.

The disclosed methods of site selection and dermal excitation increase accuracy and improve patient comfort in comparison to existing measurement regimes. While a measurement protocol permitting the use, at any time, of alternative-site measurement techniques (either blood-drawing or noninvasive) may reduce the subject's discomfort, the methods disclosed above are more accurate due to their designation of measurement site during the restricted period. This is a surprising result, especially where a noninvasive infrared monitor is used, as one skilled in the art would expect accuracy to decrease when measuring through the stratum corneum layer of skin at the fingertips, which is thicker than that found at the forearm. Where blood-drawing measurement techniques must be used, the disclosed method is also less painful for a subject than known “fingertiponly” measurement protocols, as the frequency of fingertip measurements is kept to a minimum while preserving accuracy. This too is a significant result, as previous methods did not provide for any indication of when alternative-site measurements may be permitted, much less an indication properly timed to preserve accuracy of the individual measurements. However, it will be appreciated that some embodiments also encompass measurements taken only on the on-site locations, especially measurements taken noninvasively.

In one embodiment, there is provided a method of determining a location on a subject's body whereat analyte measurements may be taken, based on the amount of elapsed time after the subject has eaten. The method comprises selecting an on-site location and an alternative site, and the on-site location and the alternative site comprise distinct areas on the subject's body. The method further comprises establishing a relationship between a restricted time period and the on-site location and between an unrestricted time period and the alternative site, the restricted time period commencing immediately after the subject eats, the unrestricted time period commencing immediately after the restricted time period terminates. The method further comprises determining whether the amount of elapsed time after the subject has eaten falls within during the restricted time period, and restricting the subject to taking analyte measurements at the on-site location during the restricted time period.

In another embodiment, there is provided a method of measuring analyte concentration within the living tissue of a subject at a measurement location on the body of the subject. The method comprises designating a restricted time period and an unrestricted time period, the restricted time period commencing immediately after the subject eats, the unrestricted time period commencing immediately after the restricted time period terminates. The method further comprises selecting only an on-site measurement location during a restricted time period, and selecting any of an on-site measurement location and an alternative-site measurement location during an unrestricted time period.

### BRIEF DESCRIPTION OF THE DRAWINGS

FIG. 1 is a schematic view of a noninvasive optical detection system.

FIG. 2 is a perspective view of a window assembly for use with the noninvasive detection system.

FIG. 3 is an exploded schematic view of an alternative window assembly for use with the noninvasive detection system.

FIG. 4 is a plan view of the window assembly connected to a cooling system.

FIG. 5 is a plan view of the window assembly connected to a cold reservoir.

FIG. 6 is a cutaway view of a heat sink for use with the noninvasive detection system.

FIG. 6A is a cutaway perspective view of a lower portion of the noninvasive detection system of FIG. 1.

FIG. 7 is a schematic view of a control system for use with the noninvasive optical detection system.

FIG. 8 depicts a first methodology for determining the concentration of an analyte of interest.

FIG. 9 depicts a second methodology for determining the concentration of an analyte of interest.

FIG. 10 depicts a third methodology for determining the concentration of an analyte of interest.

FIG. 11 depicts a fourth methodology for determining the concentration of an analyte of interest.

FIG. 12 depicts a fifth methodology for determining the concentration of an analyte of interest.

FIG. 13 is a schematic view of a reagentless whole-blood detection system.

FIG. 14 is a perspective view of one embodiment of a cuvette for use with the reagentless whole-blood detection system.

FIG. 15 is a plan view of another embodiment of a cuvette for use with the reagentless whole-blood detection system.

FIG. 16 is a disassembled plan view of the cuvette shown in FIG. 15.

FIG. 16A is an exploded perspective view of the cuvette of FIG. 15.

FIG. 17 is a side view of the cuvette of FIG. 15.

FIG. 17A is a flowchart depicting one embodiment of a method for increasing the accuracy of an analyte concentration measurement.

FIG. 17B is a flowchart illustrating another embodiment of a method for increasing the accuracy of an analyte concentration measurement.

FIG. 18 is a table illustrating a relationship between time periods after a subject eats and site locations on the subject's body at which analyte concentration measurements may be taken.

FIG. 19 is a flow chart illustrating one embodiment of an analysis procedure whereby analyte concentration measurements are taken at a suitable site on a subject's body based on the amount of elapsed time after the subject has eaten.

FIG. 20 is a flow chart illustrating another embodiment of an analysis procedure whereby analyte concentration measurements are taken at a suitable site on a subject's body based on the amount of elapsed time after the subject has eaten.

FIG. 21 is a perspective view of one embodiment of a mechanical stabilization device.

FIG. 22 illustrates the mechanical stabilization device of FIG. 21 in an exemplifying use environment wherein the device stabilizes measurement sites on a subject's forearm and finger for determination of analyte concentration within the subject's skin.

FIG. 23 is a perspective view of another embodiment of a stabilization device, illustrated in an exemplifying use environment, wherein a first wearable window is fastened to a forearm and is in electrical communication with a second wearable window which is fastened to a finger.

FIG. 24 is a perspective view of one embodiment of a wearable window.

FIG. 24A is an exploded view of the wearable window of FIG. 24.

FIG. 24B illustrates one embodiment of an electrical connection established between the wearable window of FIG. 24 and an optical measurement system.

FIG. 25 is a perspective view of another embodiment of a stabilization device, illustrated in an exemplifying use environment, wherein a first site selector is fastened to a forearm and a second site selector is fastened to a finger.

FIG. 25A is a perspective view of one embodiment of a site selector.

FIG. 25B is a side elevation view of the site selector of FIG. 25A.

FIG. 25C is a top view of the site selector of FIG. 25B, taken along line 25C-25C.

### DETAILED DESCRIPTION OF THE PREFERRED EMBODIMENTS

Herein are disclosed methods and apparatus relating to the selection of a site at which are taken measurements of the concentration of an analyte within a material sample. Part I describes the measurement of an analyte by using systems such as a noninvasive analyte detection system or a whole blood analyte detection system. In one embodiment, the analyte concentration measurement system measures the concentration of glucose in blood. Part I describes methods and apparatus relating to the selection of a site at which are taken measurements of the concentration of an analyte within a material sample. In one embodiment, a restricted period commences after a subject eats, and the subject is permitted to take measurements only at “on-site” locations during the restricted period. When a restricted period is not in effect, the subject may take measurements at on-site or offsite locations.

Although certain preferred embodiments and examples are disclosed below, it will be understood by those skilled in the art that the invention extends beyond the specifically disclosed embodiments to other alternative embodiments and/or uses of the invention and obvious modifications and equivalents thereof. Thus, it is intended that the scope of the invention herein disclosed should not be limited by the particular disclosed embodiments described below.

### I. Overview of Analyte Detection Systems

Disclosed herein are analyte detection systems, including a noninvasive system discussed largely in part A below and a whole-blood system discussed largely in part B below. Also disclosed are various methods, including methods for detecting the concentration of an analyte in a material sample. The noninvasive system/method and the whole-blood system/method are related in that they both can employ optical measurement. As used herein with reference to measurement apparatus and methods, “optical” is a broad term and is used in its ordinary sense and refers, without limitation, to identification of the presence or concentration of an analyte in a material sample without requiring a chemical reaction to take place. As discussed in more detail below, the two approaches each can operate independently to perform an optical analysis of a material sample. The two approaches can also be combined in an apparatus, or the two approaches can be used together to perform different steps of a method.

In one embodiment, the two approaches are combined to perform calibration of an apparatus, e.g., of an apparatus that employs a noninvasive approach. In another embodiment, an advantageous combination of the two approaches performs an invasive measurement to achieve greater accuracy and a whole-blood measurement to minimize discomfort to the patient. For example, the whole-blood technique may be more accurate than the noninvasive technique at certain times of the day, e.g., at certain times after a meal has been consumed, or after a drug has been administered.

It should be understood, however, that any of the disclosed devices may be operated in accordance with any suitable detection methodology, and that any disclosed method may be employed in the operation of any suitable device. Furthermore, the disclosed devices and methods are applicable in a wide variety of situations or modes of operation, including but not limited to invasive, noninvasive, intermittent or continuous measurement, subcutaneous implantation, wearable detection systems, or any combination thereof.

Any method which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the method(s) in question.

A. Noninvasive System

1. Monitor Structure

FIG. 1 depicts a noninvasive optical detection system (hereinafter “noninvasive system”) 10 in a presently preferred configuration. The depicted noninvasive system 10 is particularly suited for noninvasively detecting the concentration of an analyte in a material sample S, by observing the infrared energy emitted by the sample, as will be discussed in further detail below.

As used herein, the term “noninvasive” is a broad term and is used in its ordinary sense and refers, without limitation, to analyte detection devices and methods which have the capability to determine the concentration of an analyte in in-vivo tissue samples or bodily fluids. It should be understood, however, that the noninvasive system 10 disclosed herein is not limited to noninvasive use, as the noninvasive system 10 may be employed to analyze an in-vitro fluid or tissue sample which has been obtained invasively or noninvasively. As used herein, the term “invasive” is a broad term and is used in its ordinary sense and refers, without limitation, to analyte detection methods which involve the removal of fluid samples through the skin. As used herein, the term “material sample” is a broad term and is used in its ordinary sense and refers, without limitation, to any collection of material which is suitable for analysis by the noninvasive system 10. For example, the material sample S may comprise a tissue sample, such as a human forearm, placed against the noninvasive system 10. The material sample S may also comprise a volume of a bodily fluid, such as whole blood, blood component(s), interstitial fluid or intercellular fluid obtained invasively, or saliva or urine obtained noninvasively, or any collection of organic or inorganic material. As used herein, the term “analyte” is a broad term and is used in its ordinary sense and refers, without limitation, to any chemical species the presence or concentration of which is sought in the material sample S by the noninvasive system 10. For example, the analyte(s) which may be detected by the noninvasive system 10 include but not are limited to glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, electrolytes, sodium, potassium, chloride, bicarbonate, and hormones. As used herein to describe measurement techniques, the term “continuous” is a broad term and is used in its ordinary sense and refers, without limitation, to the taking of discrete measurements more frequently than about once every 10 minutes, and/or the taking of a stream or series of measurements or other data over any suitable time interval, for example, over an interval of one to several seconds, minutes, hours, days, or longer. As used herein to describe measurement techniques, the term “intermittent” is a broad term and is used in its ordinary sense and refers, without limitation, to the taking of measurements less frequently than about once every 10 minutes.

The noninvasive system 10 preferably comprises a window assembly 12, although in some embodiments the window assembly 12 may be omitted. One function of the window assembly 12 is to permit infrared energy E to enter the noninvasive system 10 from the sample S when it is placed against an upper surface 12a of the window assembly 12. The window assembly 12 includes a heater layer (see discussion below) which is employed to heat the material sample S and stimulate emission of infrared energy therefrom. A cooling system 14, preferably comprising a Peltier-type thermoelectric device, is in thermally conductive relation to the window assembly 12 so that the temperature of the window assembly 12 and the material sample S can be manipulated in accordance with a detection methodology discussed in greater detail below. The cooling system 14 includes a cold surface 14a which is in thermally conductive relation to a cold reservoir 16 and the window assembly 12, and a hot surface 14b which is in thermally conductive relation to a heat sink 18.

As the infrared energy E enters the noninvasive system 10, it first passes through the window assembly 12, then through an optical mixer 20, and then through a collimator 22. The optical mixer 20 preferably comprises a light pipe having highly reflective inner surfaces which randomize the directionality of the infrared energy E as it passes therethrough and reflects against the mixer walls. The collimator 22 also comprises a light pipe having highly-reflective inner walls, but the walls diverge as they extend away from the mixer 20. The divergent walls cause the infrared energy E to tend to straighten as it advances toward the wider end of the collimator 22, due to the angle of incidence of the infrared energy when reflecting against the collimator walls.

From the collimator 22 the infrared energy E passes through an array of filters 24, each of which allows only a selected wavelength or band of wavelengths to pass therethrough. These wavelengths/bands are selected to highlight or isolate the absorptive effects of the analyte of interest in the detection methodology discussed in greater detail below. Each filter 24 is preferably in optical communication with a concentrator 26 and an infrared detector 28. The concentrators 26 have highly reflective, converging inner walls which concentrate the infrared energy as it advances toward the detectors 28, increasing the density of the energy incident upon the detectors 28.

The detectors 28 are in electrical communication with a control system 30 which receives electrical signals from the detectors 28 and computes the concentration of the analyte in the sample S. The control system 30 is also in electrical communication with the window 12 and cooling system 14, so as to monitor the temperature of the window 12 and/or cooling system 14 and control the delivery of electrical power to the window 12 and cooling system 14.

a. Window Assembly

A preferred configuration of the window assembly 12 is shown in perspective, as viewed from its underside (in other words, the side of the window assembly 12 opposite the sample S), in FIG. 2. The window assembly 12 generally comprises a main layer 32 formed of a highly infrared-transmissive material and a heater layer 34 affixed to the underside of the main layer 32. The main layer 32 is preferably formed from diamond, most preferably from chemical-vapor-deposited (“CVD”) diamond, with a preferred thickness of about 0.25 millimeters. In other embodiments alternative materials which are highly infrared-transmissive, such as silicon or germanium, may be used in forming the main layer 32.

The heater layer 34 preferably comprises bus bars 36 located at opposing ends of an array of heater elements 38. The bus bars 36 are in electrical communication with the elements 38 so that, upon connection of the bus bars 36 to a suitable electrical power source (not shown) a current may be passed through the elements 38 to generate heat in the window assembly 12. The heater layer 34 may also include one or more temperature sensors (not shown), such as thermistors or resistance temperature devices (RTDs), to measure the temperature of the window assembly 12 and provide temperature feedback to the control system 30 (see FIG. 1).

Still referring to FIG. 2, the heater layer 34 preferably comprises a first adhesion layer of gold or platinum (hereinafter referred to as the “gold” layer) deposited over an alloy layer which is applied to the main layer 32. The alloy layer comprises a material suitable for implementation of the heater layer 34, such as, by way of example, 10/90 titanium/tungsten, titanium/platinum, nickel/chromium, or other similar material. The gold layer preferably has a thickness of about 4000 Å, and the alloy layer preferably has a thickness ranging between about 300 Å and about 500 Å. The gold layer and/or the alloy layer may be deposited onto the main layer 32 by chemical deposition including, but not necessarily limited to, vapor deposition, liquid deposition, plating, laminating, casting, sintering, or other forming or deposition methodologies well known to those or ordinary skill in the art. If desired, the heater layer 34 may be covered with an electrically insulating coating which also enhances adhesion to the main layer 32. One preferred coating material is aluminum oxide. Other acceptable materials include, but are not limited to, titanium dioxide or zinc selenide.

The heater layer 34 may incorporate a variable pitch distance between centerlines of adjacent heater elements 38 to maintain a constant power density, and promote a uniform temperature, across the entire layer 34. Where a constant pitch distance is employed, the preferred distance is at least about 50-100 microns. Although the heater elements 38 generally have a preferred width of about 25 microns, their width may also be varied as needed for the same reasons stated above.

Alternative structures suitable for use as the heater layer 34 include, but are not limited to, thermoelectric heaters, radiofrequency (RF) heaters, infrared radiation heaters, optical heaters, heat exchangers, electrical resistance heating grids, wire bridge heating grids, or laser heaters. Whichever type of heater layer is employed, it is preferred that the heater layer obscures about 10% or less of the window assembly 12.

In a preferred embodiment, the window assembly 12 comprises substantially only the main layer 32 and the heater layer 34. Thus, when installed in an optical detection system such as the noninvasive system 10 shown in FIG. 1, the window assembly 12 will facilitate a minimally obstructed optical path between a (preferably flat) upper surface 12a of the window assembly 12 and the infrared detectors 28 of the noninvasive system 10. The optical path 32 in the preferred noninvasive system 10 proceeds only through the main layer 32 and heater layer 34 of the window assembly 12 (including any antireflective, index-matching, electrical insulating or protective coatings applied thereto or placed therein), through the optical mixer 20 and collimator 22 and to the detectors 28.

FIG. 3 depicts an exploded side view of an alternative configuration for the window assembly 12, which may be used in place of the configuration shown in FIG. 2. The window assembly 12 depicted in FIG. 3 includes near its upper surface (the surface intended for contact with the sample S) a highly infrared-transmissive, thermally conductive spreader layer 42. Underlying the spreader layer 42 is a heater layer 44. A thin electrically insulating layer (not shown), such as layer of aluminum oxide, titanium dioxide or zinc selenide, may be disposed between the heater layer 44 and the spreader layer 42. (An aluminum oxide layer also increases adhesion of the heater layer 44 to the spreader layer 42.) Adjacent to the heater layer 44 is a thermal insulating and impedance matching layer 46. Adjacent to the thermal insulating layer 46 is a thermally conductive inner layer 48. The spreader layer 42 is coated on its top surface with a thin layer of protective coating 50. The bottom surface of the inner layer 48 is coated with a thin overcoat layer 52. Preferably, the protective coating 50 and the overcoat layer 52 have antireflective properties.

The spreader layer 42 is preferably formed of a highly infrared-transmissive material having a high thermal conductivity sufficient to facilitate heat transfer from the heater layer 44 uniformly into the material sample S when it is placed against the window assembly 12. Other effective materials include, but are not limited to, CVD diamond, diamondlike carbon, gallium arsenide, germanium, and other infrared-transmissive materials having sufficiently high thermal conductivity. Preferred dimensions for the spreader layer 42 are about one inch in diameter and about 0.010 inch thick. As shown in FIG. 3, a preferred embodiment of the spreader layer 42 incorporates a beveled edge. Although not required, an approximate 45-degree bevel is preferred.

The protective layer 50 is intended to protect the top surface of the spreader layer 42 from damage. Ideally, the protective layer is highly infrared-transmissive and highly resistant to mechanical damage, such as scratching or abrasion. It is also preferred that the protective layer 50 and the overcoat layer 52 have high thermal conductivity and antireflective and/or index-matching properties. A satisfactory material for use as the protective layer 50 and the overcoat layer 52 is the multi-layer Broad Band Anti-Reflective Coating produced by Deposition Research Laboratories, Inc. of St. Charles, Mo. Diamondlike carbon coatings are also suitable.

Except as noted below, the heater layer 44 is generally similar to the heater layer 34 employed in the window assembly shown in FIG. 2. Alternatively, the heater layer 44 may comprise a doped infrared-transmissive material, such as a doped silicon layer, with regions of higher and lower resistivity. The heater layer 44 preferably has a resistance of about 2 ohms and has a preferred thickness of about 1,500 angstroms. A preferred material for forming the heater layer 44 is a gold alloy, but other acceptable materials include, but are not limited to, platinum, titanium, tungsten, copper, and nickel.

The thermal insulating layer 46 prevents the dissipation of heat from the heater element 44 while allowing the cooling system 14 to effectively cool the material sample S (see FIG. 1). This layer 46 comprises a material having thermally insulative (e.g., lower thermal conductivity than the spreader layer 42) and infrared transmissive qualities. A preferred material is a germanium-arsenic-selenium compound of the calcogenide glass family known as AMTIR-1 produced by Amorphous Materials, Inc. of Garland, Tex. The pictured embodiment has a diameter of about 0.85 inches and a preferred thickness in the range of about 0.005 to about 0.010 inches. As heat generated by the heater layer 44 passes through the spreader layer 42 into the material sample S, the thermal insulating layer 46 insulates this heat.

The inner layer 48 is formed of thermally conductive material, preferably crystalline silicon formed using a conventional floatzone crystal growth method. The purpose of the inner layer 48 is to serve as a cold-conducting mechanical base for the entire layered window assembly.

The overall optical transmission of the window assembly 12 shown in FIG. 3 is preferably at least 70%. The window assembly 12 of FIG. 3 is preferably held together and secured to the noninvasive system 10 by a holding bracket (not shown). The bracket is preferably formed of a glass-filled plastic, for example Ultem 2300, manufactured by General Electric. Ultem 2300 has low thermal conductivity which prevents heat transfer from the layered window assembly 12.

b. Cooling System

The cooling system 14 (see FIG. 1) preferably comprises a Peltier-type thermoelectric device. Thus, the application of an electrical current to the preferred cooling system 14 causes the cold surface 14a to cool and causes the opposing hot surface 14b to heat up. The cooling system 14 cools the window assembly 12 via the situation of the window assembly 12 in thermally conductive relation to the cold surface 14a of the cooling system 14. It is contemplated that the cooling system 14, the heater layer 34, or both, can be operated to induce a desired time-varying temperature in the window assembly 12 to create an oscillating thermal gradient in the sample S, in accordance with various analyte-detection methodologies discussed herein.

Preferably, the cold reservoir 16 is positioned between the cooling system 14 and the window assembly 12, and functions as a thermal conductor between the system 14 and the window assembly 12. The cold reservoir 16 is formed from a suitable thermally conductive material, preferably brass. Alternatively, the window assembly 12 can be situated in direct contact with the cold surface 14a of the cooling system 14.

In alternative embodiments, the cooling system 14 may comprise a heat exchanger through which a coolant, such as air, nitrogen or chilled water, is pumped, or a passive conduction cooler such as a heat sink. As a further alternative, a gas coolant such as nitrogen may be circulated through the interior of the noninvasive system 10 so as to contact the underside of the window assembly 12 (see FIG. 1) and conduct heat therefrom.

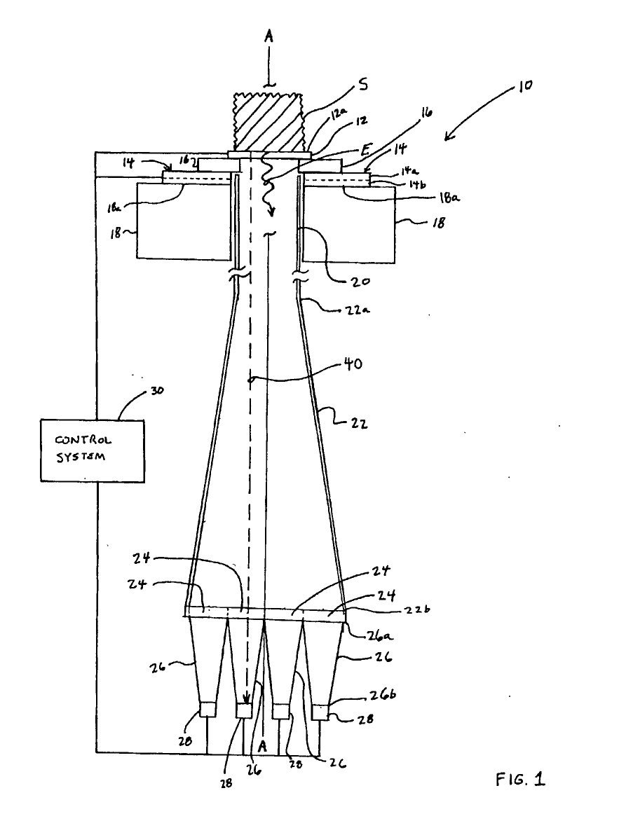

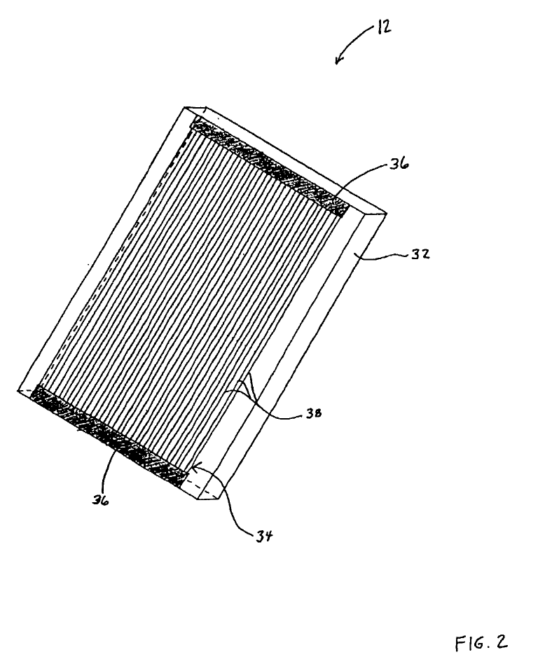

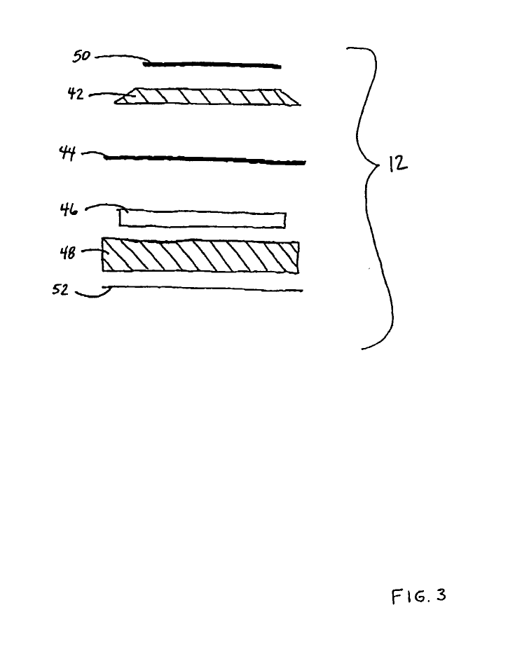

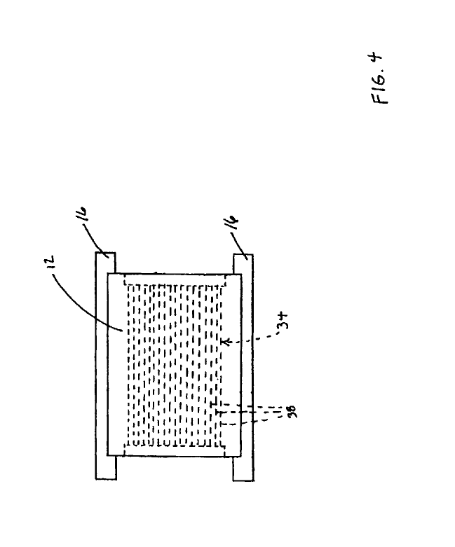

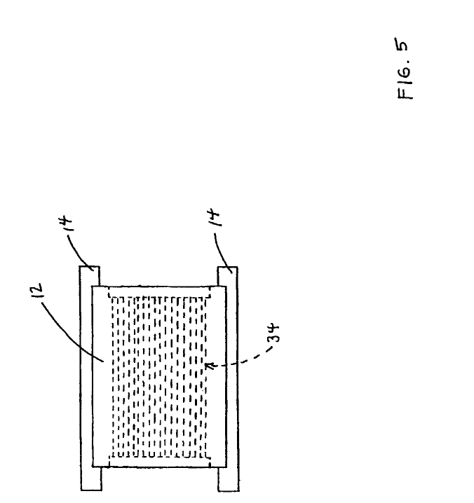

FIG. 4 is a top schematic view of a preferred arrangement of the window assembly 12 (of the type shown in FIG. 2) and the cold reservoir 16, and FIG. 5 is a top schematic view of an alternative arrangement in which the window assembly 12 directly contacts the cooling system 14. The cold reservoir 16/cooling system 14 preferably contacts the underside of the window assembly 12 along opposing edges thereof, on either side of the heater layer 34. With thermal conductivity thus established between the window assembly 12 and the cooling system 14, the window assembly can be cooled as needed during operation of the noninvasive system 10. In order to promote a substantially uniform or isothermal temperature profile over the upper surface of the window assembly 12, the pitch distance between centerlines of adjacent heater elements 38 may be made smaller (thereby increasing the density of heater elements 38) near the region(s) of contact between the window assembly 12 and the cold reservoir 16/cooling system 14. As a supplement or alternative, the heater elements 38 themselves may be made wider near these regions of contact. As used herein, “isothermal” is a broad term and is used in its ordinary sense and refers, without limitation, to a condition in which, at a given point in time, the temperature of the window assembly 12 or other structure is substantially uniform across a surface intended for placement in thermally conductive relation to the material sample S. Thus, although the temperature of the structure or surface may fluctuate over time, at any given point in time the structure or surface may nonetheless be isothermal.

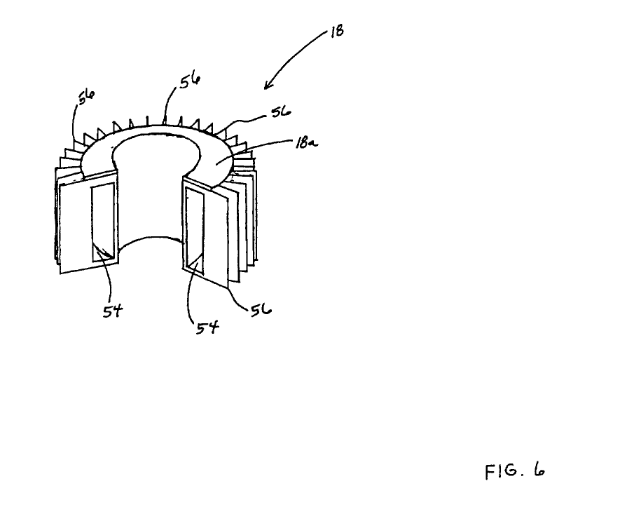

The heat sink 18 drains waste heat from the hot surface 14b of the cooling system 16 and stabilizes the operational temperature of the noninvasive system 10. The preferred heat sink 18 (see FIG. 6) comprises a hollow structure formed from brass or any other suitable material having a relatively high specific heat and high heat conductivity. The heat sink 18 has a conduction surface 18a which, when the heat sink 18 is installed in the noninvasive system 18, is in thermally conductive relation to the hot surface 14b of the cooling system 14 (see FIG. 1). A cavity 54 is formed in the heat sink 18 and preferably contains a phase-change material (not shown) to increase the capacity of the sink 18. A preferred phase change material is a hydrated salt, such as calciumchloride hexahydrate, available under the name TH29 from PCM Thermal Solutions, Inc., of Naperville, Ill. Alternatively, the cavity 54 may be omitted to create a heat sink 18 comprising a solid, unitary mass. The heat sink 18 also forms a number of fins 56 to further increase the conduction of heat from the sink 18 to surrounding air.

Alternatively, the heat sink 18 may be formed integrally with the optical mixer 20 and/or the collimator 22 as a unitary mass of rigid, heat-conductive material such as brass or aluminum. In such a heat sink, the mixer 20 and/or collimator 22 extend axially through the heat sink 18, and the heat sink defines the inner walls of the mixer 20 and/or collimator 22. These inner walls are coated and/or polished to have appropriate reflectivity and nonabsorbance in infrared wavelengths as will be further described below. Where such a unitary heat sink-mixer-collimator is employed, it is desirable to thermally insulate the detector array from the heat sink.

It should be understood that any suitable structure may be employed to heat and/or cool the material sample S, instead of or in addition to the window assembly 12/cooling system 14 disclosed above, so long a proper degree of cycled heating and/or cooling are imparted to the material sample S. In addition other forms of energy, such as but not limited to light, radiation, chemically induced heat, friction and vibration, may be employed to heat the material sample S. It will be further appreciated that heating of the sample can achieved by any suitable method, such as convection, conduction, radiation, etc.

C. Optics

As shown in FIG. 1, the optical mixer 20 comprises a light pipe with an inner surface coating which is highly reflective and minimally absorptive in infrared wavelengths, preferably a polished gold coating, although other suitable coatings may be used where other wavelengths of electromagnetic radiation are employed. The pipe itself may be fabricated from a another rigid material such as aluminum or stainless steel, as long as the inner surfaces are coated or otherwise treated to be highly reflective. Preferably, the optical mixer 20 has a rectangular cross-section (as taken orthogonal to the longitudinal axis A-A of the mixer 20 and the collimator 22), although other cross-sectional shapes, such as other polygonal shapes or circular or elliptical shapes, may be employed in alternative embodiments. The inner walls of the optical mixer 20 are substantially parallel to the longitudinal axis A-A of the mixer 20 and the collimator 22. The highly reflective and substantially parallel inner walls of the mixer 20 maximize the number of times the infrared energy E will be reflected between the walls of the mixer 20, thoroughly mixing the infrared energy E as it propagates through the mixer 20. In a presently preferred embodiment, the mixer 20 is about 1.2 inches to 2.4 inches in length and its cross-section is a rectangle of about 0.4 inches by about 0.6 inches. Of course, other dimensions may be employed in constructing the mixer 20. In particular it is be advantageous to miniaturize the mixer or otherwise make it as small as possible

Still referring to FIG. 1, the collimator 22 comprises a tube with an inner surface coating which is highly reflective and minimally absorptive in infrared wavelengths, preferably a polished gold coating. The tube itself may be fabricated from a another rigid material such as aluminum, nickel or stainless steel, as long as the inner surfaces are coated or otherwise treated to be highly reflective. Preferably, the collimator 22 has a rectangular cross-section, although other cross-sectional shapes, such as other polygonal shapes or circular, parabolic or elliptical shapes, may be employed in alternative embodiments. The inner walls of the collimator 22 diverge as they extend away from the mixer 20. Preferably, the inner walls of the collimator 22 are substantially straight and form an angle of about 7 degrees with respect to the longitudinal axis A-A. The collimator 22 aligns the infrared energy E to propagate in a direction that is generally parallel to the longitudinal axis A-A of the mixer 20 and the collimator 22, so that the infrared energy E will strike the surface of the filters 24 at an angle as close to 90 degrees as possible.

In a presently preferred embodiment, the collimator is about 7.5 inches in length. At its narrow end 22a, the cross-section of the collimator 22 is a rectangle of about 0.4 inches by 0.6 inches. At its wide end 22b, the collimator 22 has a rectangular cross-section of about 1.8 inches by 2.6 inches. Preferably, the collimator 22 aligns the infrared energy E to an angle of incidence (with respect to the longitudinal axis A-A) of about 0-15 degrees before the energy E impinges upon the filters 24. Of course, other dimensions or incidence angles may be employed in constructing and operating the collimator 22.

With further reference to FIGS. 1 and 6A, each concentrator 26 comprises a tapered surface oriented such that its wide end 26a is adapted to receive the infrared energy exiting the corresponding filter 24, and such that its narrow end 26b is adjacent to the corresponding detector 28. The inward-facing surfaces of the concentrators 26 have an inner surface coating which is highly reflective and minimally absorptive in infrared wavelengths, preferably a polished gold coating. The concentrators 26 themselves may be fabricated from a another rigid material such as aluminum, nickel or stainless steel, so long as their inner surfaces are coated or otherwise treated to be highly reflective.

Preferably, the concentrators 26 have a rectangular cross-section (as taken orthogonal to the longitudinal axis A-A), although other cross-sectional shapes, such as other polygonal shapes or circular, parabolic or elliptical shapes, may be employed in alternative embodiments. The inner walls of the concentrators converge as they extend toward the narrow end 26b. Preferably, the inner walls of the collimators 26 are substantially straight and form an angle of about 8 degrees with respect to the longitudinal axis A-A. Such a configuration is adapted to concentrate infrared energy as it passes through the concentrators 26 from the wide end 26a to the narrow end 26b, before reaching the detectors 28.

In a presently preferred embodiment, each concentrator 26 is about 1.5 inches in length. At the wide end 26a, the cross-section of each concentrator 26 is a rectangle of about 0.6 inches by 0.57 inches. At the narrow end 26b, each concentrator 26 has a rectangular cross-section of about 0.177 inches by 0.177 inches. Of course, other dimensions or incidence angles may be employed in constructing the concentrators 26.

d. Filters

The filters 24 preferably comprise standard interference-type infrared filters, widely available from manufacturers such as Optical Coating Laboratory, Inc. (“OCLI”) of Santa Rosa, Calif. In the embodiment illustrated in FIG. 1, a 3×4 array of filters 24 is positioned above a 3×4 array of detectors 28 and concentrators 26. As employed in this embodiment, the filters 24 are arranged in four groups of three filters having the same wavelength sensitivity. These four groups have bandpass center wavelengths of 7.15 μm±0.03 μm, 8.40 μm±0.03 μm, 9.48 μm±0.04 μm, and 11.10 μm±0.04 μm, respectively, which correspond to wavelengths around which water and glucose absorb electromagnetic radiation. Typical bandwidths for these filters range from 0.20 μm to 0.50 μm.

In an alternative embodiment, the array of wavelength-specific filters 24 may be replaced with a single Fabry-Perot interferometer, which can provide wavelength sensitivity which varies as a sample of infrared energy is taken from the material sample S. Thus, this embodiment permits the use of only one detector 28, the output signal of which varies in wavelength specificity over time. The output signal can be de-multiplexed based on the wavelength sensitivities induced by the Fabry-Perot interferometer, to provide a multiple-wavelength profile of the infrared energy emitted by the material sample S. In this embodiment, the optical mixer 20 may be omitted, as only one detector 28 need be employed.

In still other embodiments, the array of filters 24 may comprise a filter wheel that rotates different filters with varying wavelength sensitivities over a single detector 24. Alternatively, an electronically tunable infrared filter may be employed in a manner similar to the Fabry-Perot interferometer discussed above, to provide wavelength sensitivity which varies during the detection process. In either of these embodiments, the optical mixer 20 may be omitted, as only one detector 28 need be employed.

e. Detectors

The detectors 28 may comprise any detector type suitable for sensing infrared energy, preferably in the mid-infrared wavelengths. For example, the detectors 28 may comprise mercury-cadmium-telluride (MCT) detectors. A detector such as a Fermionics (Simi Valley, Calif.) model PV-9.1 with a PVA481-1 pre-amplifier is acceptable. Similar units from other manufacturers such as Graseby (Tampa, Fla.) can be substituted. Other suitable components for use as the detectors 28 include pyroelectric detectors, thermopiles, bolometers, silicon microbolometers and lead-salt focal plane arrays.

f. Control System

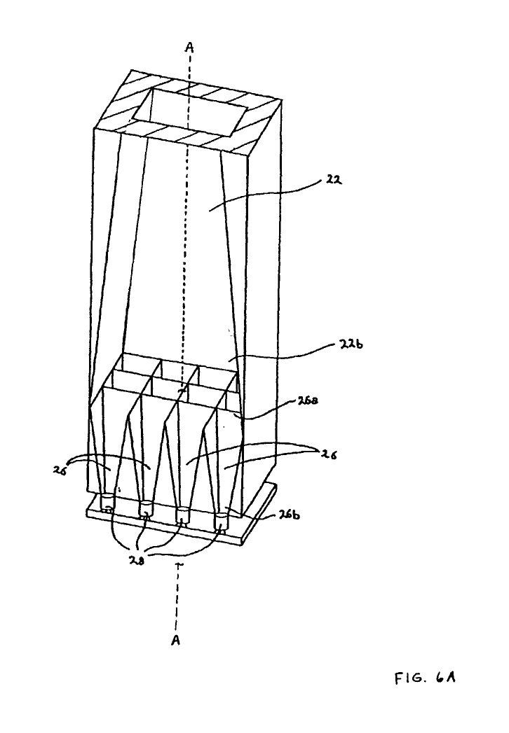

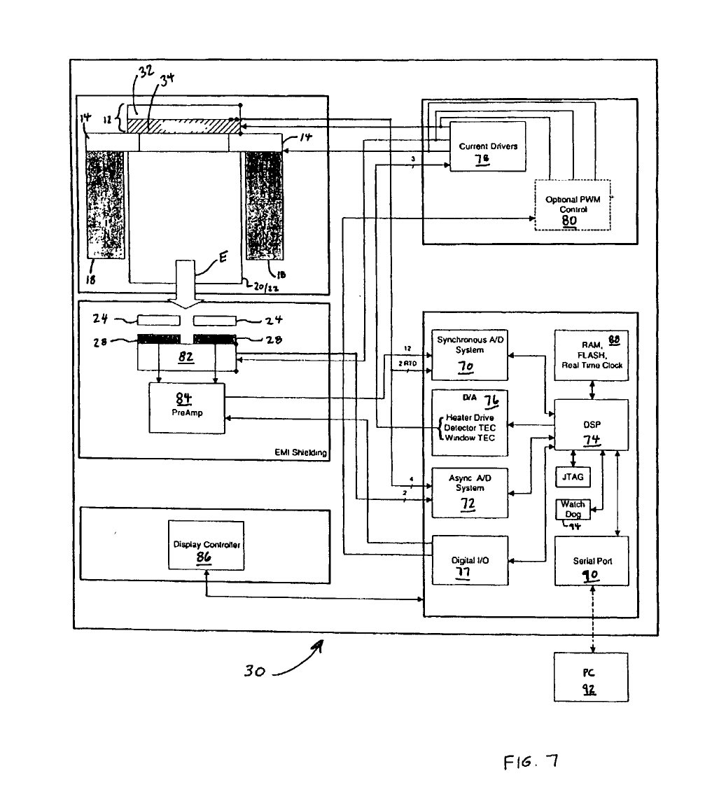

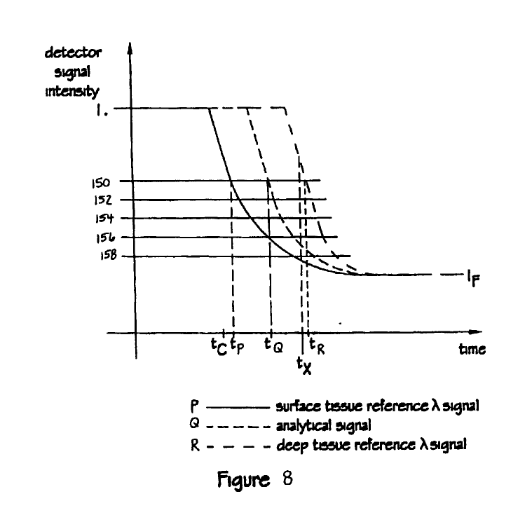

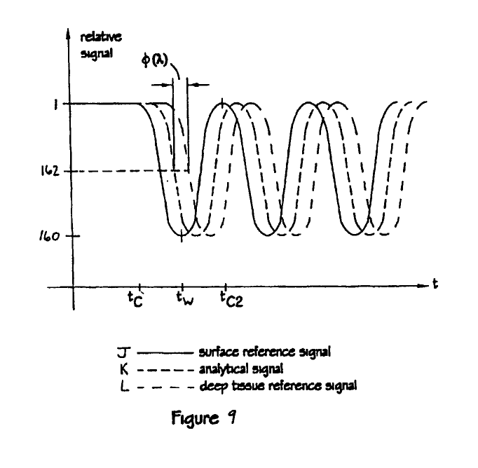

FIG. 7 depicts the control system 30 in greater detail, as well as the interconnections between the control system and other relevant portions of the noninvasive system. The control system includes a temperature control subsystem and a data acquisition subsystem.

In the temperature control subsystem, temperature sensors (such as RTDs and/or thermistors) located in the window assembly 12 provide a window temperature signal to a synchronous analog-to-digital conversion system 70 and an asynchronous analog-to-digital conversion system 72. The A/D systems 70, 72 in turn provide a digital window temperature signal to a digital signal processor (DSP) 74. The processor 74 executes a window temperature control algorithm and determines appropriate control inputs for the heater layer 34 of the window assembly 12 and/or for the cooling system 14, based on the information contained in the window temperature signal. The processor 74 outputs one or more digital control signals to a digital-to-analog conversion system 76 which in turn provides one or more analog control signals to current drivers 78. In response to the control signal(s), the current drivers 78 regulate the power supplied to the heater layer 34 and/or to the cooling system 14. In one embodiment, the processor 74 provides a control signal through a digital I/O device 77 to a pulse-width modulator (PWM) control 80, which provides a signal that controls the operation of the current drivers 78. Alternatively, a low-pass filter (not shown) at the output of the PWM provides for continuous operation of the current drivers 78.

In another embodiment, temperature sensors may be located at the cooling system 14 and appropriately connected to the A/D system(s) and processor to provide closed-loop control of the cooling system as well.

In yet another embodiment, a detector cooling system 82 is located in thermally conductive relation to one or more of the detectors 28. The detector cooling system 82 may comprise any of the devices disclosed above as comprising the cooling system 14, and preferably comprises a Peltier-type thermoelectric device. The temperature control subsystem may also include temperature sensors, such as RTDs and/or thermistors, located in or adjacent to the detector cooling system 82, and electrical connections between these sensors and the asynchronous A/D system 72. The temperature sensors of the detector cooling system 82 provide detector temperature signals to the processor 74. In one embodiment, the detector cooling system 82 operates independently of the window temperature control system, and the detector cooling system temperature signals are sampled using the asynchronous A/D system 72. In accordance with the temperature control algorithm, the processor 74 determines appropriate control inputs for the detector cooling system 82, based on the information contained in the detector temperature signal. The processor 74 outputs digital control signals to the D/A system 76 which in turn provides analog control signals to the current drivers 78. In response to the control signals, the current drivers 78 regulate the power supplied to the detector cooling system 14. In one embodiment, the processor 74 also provides a control signal through the digital I/O device 77 and the PWM control 80, to control the operation of the detector cooling system 82 by the current drivers 78. Alternatively, a low-pass filter (not shown) at the output of the PWM provides for continuous operation of the current drivers 78.

In the data acquisition subsystem, the detectors 28 respond to the infrared energy E incident thereon by passing one or more analog detector signals to a preamp 84. The preamp 84 amplifies the detector signals and passes them to the synchronous A/D system 70, which converts the detector signals to digital form and passes them to the processor 74. The processor 74 determines the concentrations of the analyte(s) of interest, based on the detector signals and a concentration-analysis algorithm and/or phase/concentration regression model stored in a memory module 88. The concentration-analysis algorithm and/or phase/concentration regression model may be developed according to any of the analysis methodologies discussed herein. The processor may communicate the concentration results and/or other information to a display controller 86, which operates a display (not shown), such as an LCD display, to present the information to the user.

A watchdog timer 94 may be employed to ensure that the processor 74 is operating correctly. If the watchdog timer 94 does not receive a signal from the processor 74 within a specified time, the watchdog timer 94 resets the processor 74. The control system may also include a JTAG interface 96 to enable testing of the noninvasive system 10.

In one embodiment, the synchronous A/D system 70 comprises a 20-bit, 14 channel system, and the asynchronous A/D system 72 comprises a 16-bit, 16 channel system. The preamp may comprise a 12-channel preamp corresponding to an array of 12 detectors 28.

The control system may also include a serial port 90 or other conventional data port to permit connection to a personal computer 92. The personal computer can be employed to update the algorithm(s) and/or phase/concentration regression model(s) stored in the memory module 88, or to download a compilation of analyte-concentration data from the noninvasive system. A real-time clock or other timing device may be accessible by the processor 74 to make any time-dependent calculations which may be desirable to a user.

2. Analysis Methodology

The detector(s) 28 of the noninvasive system 10 are used to detect the infrared energy emitted by the material sample S in various desired wavelengths. At each measured wavelength, the material sample S emits infrared energy at an intensity which varies over time. The time-varying intensities arise largely in response to the use of the window assembly 12 (including its heater layer 34) and the cooling system 14 to induce a thermal gradient in the material sample S. As used herein, “thermal gradient” is a broad term and is used in its ordinary sense and refers, without limitation, to a difference in temperature and/or thermal energy between different locations, such as different depths, of a material sample, which can be induced by any suitable method of increasing or decreasing the temperature and/or thermal energy in one or more locations of the sample. As will be discussed in detail below, the concentration of an analyte of interest (such as glucose) in the material sample S can be determined with a device such as the noninvasive system 10, by comparing the time-varying intensity profiles of the various measured wavelengths.

Analysis methodologies are discussed herein within the context of detecting the concentration of glucose within a material sample, such as a tissue sample, which includes a large proportion of water. However, it will evident that these methodologies are not limited to this context and may be applied to the detection of a wide variety of analytes within a wide variety of sample types. It should also be understood that other suitable analysis methodologies and suitable variations of the disclosed methodologies may be employed in operating an analyte detection system, such as the noninvasive system 10.

As shown in FIG. 8, a first reference signal P may be measured at a first reference wavelength. The first reference signal P is measured at a wavelength where water strongly absorbs (e.g., 2.9 μm or 6.1 μm). Because water strongly absorbs radiation at these wavelengths, the detector signal intensity is reduced at those wavelengths. Moreover, at these wavelengths water absorbs the photon emissions emanating from deep inside the sample. The net effect is that a signal emitted at these wavelengths from deep inside the sample is not easily detected. The first reference signal P is thus a good indicator of thermal-gradient effects near the sample surface and may be known as a surface reference signal. This signal may be calibrated and normalized, in the absence of heating or cooling applied to the sample, to a baseline value of 1. For greater accuracy, more than one first reference wavelength may be measured. For example, both 2.9 μm and 6.1 μm may be chosen as first reference wavelengths.

As further shown in FIG. 8, a second reference signal R may also be measured. The second signal R may be measured at a wavelength where water has very low absorbance (e.g., 3.6 μm or 4.2 μm). This second reference signal R thus provides the analyst with information concerning the deeper regions of the sample, whereas the first signal P provides information concerning the sample surface. This signal may also be calibrated and normalized, in the absence of heating or cooling applied to the sample, to a baseline value of 1. As with the first (surface) reference signal P, greater accuracy may be obtained by using more than one second (deep) reference signal R.

In order to determine analyte concentration, a third (analytical) signal Q is also measured. This signal is measured at an IR absorbance peak of the selected analyte. The IR absorbance peaks for glucose are in the range of about 6.5 μm to 11.0 μm. This detector signal may also be calibrated and normalized, in the absence of heating or cooling applied to the material sample S, to a baseline value of 1. As with the reference signals P, R, the analytical signal Q may be measured at more than one absorbance peak.

Optionally, or additionally, reference signals may be measured at wavelengths that bracket the analyte absorbance peak. These signals may be advantageously monitored at reference wavelengths which do not overlap the analyte absorbance peaks. Further, it is advantageous to measure reference wavelengths at absorbance peaks which do not overlap the absorbance peaks of other possible constituents contained in the sample.

a. Basic Thermal Gradient

As further shown in FIG. 8, the signal intensities P, Q, R are shown initially at the normalized baseline signal intensity of 1. This of course reflects the baseline radiative behavior of a test sample in the absence of applied heating or cooling. At a time t_{C}, the surface of the sample is subjected to a temperature event which induces a thermal gradient in the sample. The gradient can be induced by heating or cooling the sample surface. The example shown in FIG. 8 uses cooling, for example, using a 10° C. cooling event. In response to the cooling event, the intensities of the detector signals P, Q, R decrease over time.

Since the cooling of the sample is neither uniform nor instantaneous, the surface cools before the deeper regions of the sample cool. As each of the signals P, Q, R drop in intensity, a pattern emerges. Signal intensity declines as expected, but as the signals P, Q, R reach a given amplitude value (or series of amplitude values: 150, 152, 154, 156, 158), certain temporal effects are noted. After the cooling event is induced at t_{C}, the first (surface) reference signal P declines in amplitude most rapidly, reaching a checkpoint 150 first, at time t_{P}. This is due to the fact that the first reference signal P mirrors the sample's radiative characteristics near the surface of the sample. Since the sample surface cools before the underlying regions, the surface (first) reference signal P drops in intensity first.

Simultaneously, the second reference signal R is monitored. Since the second reference signal R corresponds to the radiation characteristics of deeper regions of the sample, which do not cool as rapidly as the surface (due to the time needed for the surface cooling to propagate into the deeper regions of the sample), the intensity of signal R does not decline until slightly later. Consequently, the signal R does not reach the magnitude 150 until some later time t_{R}. In other words, there exists a time delay between the time t_{P} at which the amplitude of the first reference signal P reaches the checkpoint 150 and the time t_{R} at which the second reference signal R reaches the same checkpoint 150. This time delay can be expressed as a phase difference Φ(λ). Additionally, a phase difference may be measured between the analytical signal Q and either or both reference signals P, R.

As the concentration of analyte increases, the amount of absorbance at the analytical wavelength increases. This reduces the intensity of the analytical signal Q in a concentration-dependent way. Consequently, the analytical signal Q reaches intensity 150 at some intermediate time t_{Q}. The higher the concentration of analyte, the more the analytical signal Q shifts to the left in FIG. 8. As a result, with increasing analyte concentration, the phase difference Φ(λ) decreases relative to the first (surface) reference signal P and increases relative to the second (deep tissue) reference signal R. The phase difference(s) Φ(λ) are directly related to analyte concentration and can be used to make accurate determinations of analyte concentration.

The phase difference Φ(λ) between the first (surface) reference signal P and the analytical signal Q is represented by the equation:

Φ(λ)=|t_{P}−t_{Q}|

The magnitude of this phase difference decreases with increasing analyte concentration.

The phase difference Φ(λ) between the second (deep tissue) reference signal R and the analytical signal Q signal is represented by the equation:

Φ(λ)=|t_{Q}−t_{R}|

The magnitude of this phase difference increases with increasing analyte concentration.

Accuracy may be enhanced by choosing several checkpoints, for example, 150, 152, 154, 156, and 158 and averaging the phase differences observed at each checkpoint. The accuracy of this method may be further enhanced by integrating the phase difference(s) continuously over the entire test period. Because in this example only a single temperature event (here, a cooling event) has been induced, the sample reaches a new lower equilibrium temperature and the signals stabilize at a new constant level IF. Of course, the method works equally well with thermal gradients induced by heating or by the application or introduction of other forms of energy, such as but not limited to light, radiation, chemically induced heat, friction and vibration.

This methodology is not limited to the determination of phase difference. At any given time (for example, at a time t_{X}) the amplitude of the analytical signal Q may be compared to the amplitude of either or both of the reference signals P, R. The difference in amplitude may be observed and processed to determine analyte concentration.

This method, the variants disclosed herein, and the apparatus disclosed as suitable for application of the method(s), are not limited to the detection of in-vivo glucose concentration. The method and disclosed variants and apparatus may be used on human, animal, or even plant subjects, or on organic or inorganic compositions in a non-medical setting. The method may be used to take measurements of in-vivo or in-vitro samples of virtually any kind. The method is useful for measuring the concentration of a wide range of additional chemical analytes, including but-not limited to, glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, hormones, as well as other chemical compounds. To detect a given analyte, one needs only to select appropriate analytical and reference wavelengths.

The method is adaptable and may be used to determine chemical concentrations in samples of body fluids (e.g., blood, urine or saliva) once they have been extracted from a patient. In fact, the method may be used for the measurement of in-vitro samples of virtually any kind.

b. Modulated Thermal Gradient

In a variation of the methodology described above, a periodically modulated thermal gradient can be employed to make accurate determinations of analyte concentration.

As previously shown in FIG. 8, once a thermal gradient is induced in the sample, the reference and analytical signals P, Q, R fall out of phase with respect to each other. This phase difference Φ(λ) is present whether the thermal gradient is induced through heating or cooling. By alternatively subjecting the test sample to cyclic pattern of heating, cooling, or alternately heating and cooling, an oscillating thermal gradient may be induced in a sample for an extended period of time.

An oscillating thermal gradient is illustrated using a sinusoidally modulated gradient. FIG. 9 depicts detector signals emanating from a test sample. As with the methodology shown in FIG. 8, one or more reference signals J, L are measured. One or more analytical signals K are also monitored. These signals may be calibrated and normalized, in the absence of heating or cooling applied to the sample, to a baseline value of 1. FIG. 9 shows the signals after normalization. At some time t_{C}, a temperature event (e.g., cooling) is induced at the sample surface. This causes a decline in the detector signal. As shown in FIG. 8, the signals (P, Q, R) decline until the thermal gradient disappears and a new equilibrium detector signal IF is reached. In the method shown in FIG. 9, as the gradient begins to disappear at a signal intensity 160, a heating event, at a time t_{W}, is induced in the sample surface. As a result the detector output signals J, K, L will rise as the sample temperature rises. At some later time t_{C2}, another cooling event is induced, causing the temperature and detector signals to decline. This cycle of cooling and heating may be repeated over a time interval of arbitrary length. Moreover, if the cooling and heating events are timed properly, a periodically modulated thermal gradient may be induced in the test sample.

As previously explained in the discussions relating to FIG. 8, the phase difference Φ(λ) may be measured and used to determine analyte concentration. FIG. 9 shows that the first (surface) reference signal J declines and rises in intensity first. The second (deep tissue) reference signal L declines and rises in a time-delayed manner relative to the first reference signal J. The analytical signal K exhibits a time/phase delay dependent on the analyte concentration. With increasing concentration, the analytical signal K shifts to the left in FIG. 9. As with FIG. 8, the phase difference Φ(λ) may be measured. For example, a phase difference Φ(λ) between the second reference signal L and the analytical signal K, may be measured at a set amplitude 162 as shown in FIG. 9. Again, the magnitude of the phase signal reflects the analyte concentration of the sample.

The phase-difference information compiled by any of the methodologies disclosed herein can correlated by the control system 30 (see FIG. 1) with previously determined phase-difference information to determine the analyte concentration in the sample. This correlation could involve comparison of the phase-difference information received from analysis of the sample, with a data set containing the phase-difference profiles observed from analysis of wide variety of standards of known analyte concentration. In one embodiment, a phase/concentration curve or regression model is established by applying regression techniques to a set of phase-difference data observed in standards of known analyte concentration. This curve is used to estimate the analyte concentration in a sample based on the phase-difference information received from the sample.

Advantageously, the phase difference Φ(λ) may be measured continuously throughout the test period. The phase-difference measurements may be integrated over the entire test period for an extremely accurate measure of phase difference Φ(λ). Accuracy may also be improved by using more than one reference signal and/or more than one analytical signal.

As an alternative or as a supplement to measuring phase difference(s), differences in amplitude between the analytical and reference signal(s) may be measured and employed to determine analyte concentration. Additional details relating to this technique and not necessary to repeat here may be found in the Assignee's U.S. patent application Ser. No. 09/538,164, incorporated by reference below.

Additionally, these methods may be advantageously employed to simultaneously measure the concentration of one or more analytes. By choosing reference and analyte wavelengths that do not overlap, phase differences can be simultaneously measured and processed to determine analyte concentrations. Although FIG. 9 illustrates the method used in conjunction with a sinusoidally modulated thermal gradient, the principle applies to thermal gradients conforming to any periodic function. In more complex cases, analysis using signal processing with Fourier transforms or other techniques allows accurate determinations of phase difference Φ(λ) and analyte concentration.

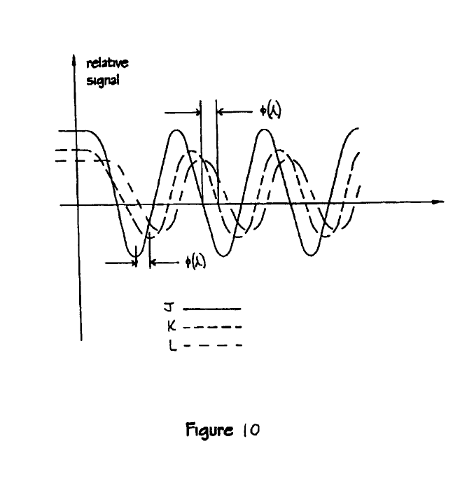

As shown in FIG. 10, the magnitude of the phase differences may be determined by measuring the time intervals between the amplitude peaks (or troughs) of the reference signals J, L and the analytical signal K. Alternatively, the time intervals between the “zero crossings” (the point at which the signal amplitude changes from positive to negative, or negative to positive) may be used to determine the phase difference between the analytical signal K and the reference signals J, L. This information is subsequently processed and a determination of analyte concentration may then be made. This particular method has the advantage of not requiring normalized signals.

As a further alternative, two or more driving frequencies may be employed to determine analyte concentrations at selected depths within the sample. A slow (e.g., 1 Hz) driving frequency creates a thermal gradient which penetrates deeper into the sample than the gradient created by a fast (e.g., 3 Hz) driving frequency. This is because the individual heating and/or cooling events are longer in duration where the driving frequency is lower. Thus, the use of a slow driving frequency provides analyte-concentration information from a deeper “slice” of the sample than does the use of a fast driving frequency.

It has been found that when analyzing a sample of human skin, a temperature event of 10° C. creates a thermal gradient which penetrates to a depth of about 150 μm, after about 500 ms of exposure. Consequently, a cooling/heating cycle or driving frequency of 1 Hz provides information to a depth of about 150 μm. It has also been determined that exposure to a temperature event of 10° C. for about 167 ms creates a thermal gradient that penetrates to a depth of about 50 μm. Therefore, a cooling/heating cycle of 3 Hz provides information to a depth of about 50 μm. By subtracting the detector signal information measured at a 3 Hz driving frequency from the detector signal information measured at a 1 Hz driving frequency, one can determine the analyte concentration(s) in the region of skin between 50 and 150 μm. Of course, a similar approach can be used to determine analyte concentrations at any desired depth range within any suitable type of sample.

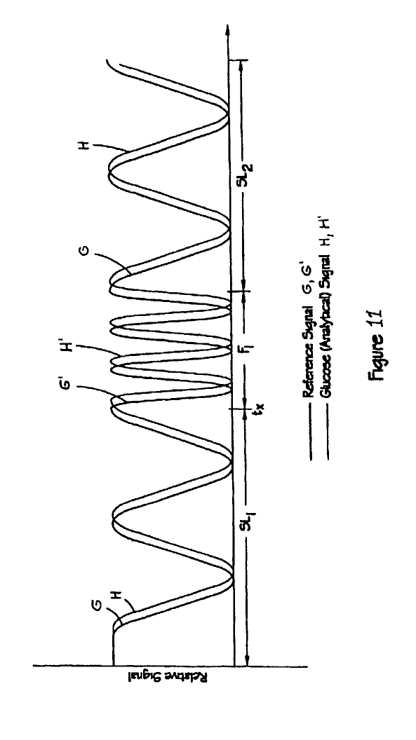

As shown in FIG. 11, alternating deep and shallow thermal gradients may be induced by alternating slow and fast driving frequencies. As with the methods described above, this variation also involves the detection and measurement of phase differences Φ(λ) between reference signals G, G′ and analytical signals H, H′. Phase differences are measured at both fast (e.g., 3 Hz) and slow (e.g., 1 Hz) driving frequencies. The slow driving frequency may continue for an arbitrarily chosen number of cycles (in region SL₁), for example, two full cycles. Then the fast driving frequency is employed for a selected duration, in region F₁. The phase difference data is compiled in the same manner as disclosed above. In addition, the fast frequency (shallow sample) phase difference data may be subtracted from the slow frequency (deep sample) data to provide an accurate determination of analyte concentration in the region of the sample between the gradient penetration depth associated with the fast driving frequency and that associated with the slow driving frequency.

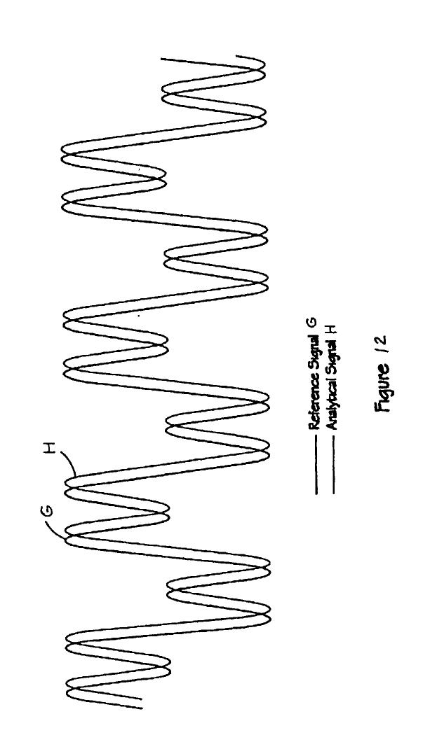

The driving frequencies (e.g., 1 Hz and 3 Hz) can be multiplexed as shown in FIG. 12. The fast (3 Hz) and slow (1 Hz) driving frequencies can be superimposed rather than sequentially implemented. During analysis, the data can be separated by frequency (using Fourier transform or other techniques) and independent measurements of phase delay at each of the driving frequencies may be calculated. Once resolved, the two sets of phase delay data are processed to determine absorbance and analyte concentration.

Additional details not necessary to repeat here may be found in U.S. Pat. No. 6,198,949, titled SOLID-STATE NON-INVASIVE INFRARED ABSORPTION SPECTROMETER FOR THE GENERATION AND CAPTURE OF THERMAL GRADIENT SPECTRA FROM LIVING TISSUE, issued Mar. 6, 2001; U.S. Pat. No. 6,161,028, titled METHOD FOR DETERMINING ANALYTE CONCENTRATION USING PERIODIC TEMPERATURE MODULATION AND PHASE DETECTION, issued Dec. 12, 2000; U.S. Pat. No. 5,877,500, titled MULTICHANNEL INFRARED DETECTOR WITH OPTICAL CONCENTRATORS FOR EACH CHANNEL, issued on Mar. 2, 1999; U.S. patent application Ser. No. 09/538,164, filed Mar. 30, 2000 and titled METHOD AND APPARATUS FOR DETERMINING ANALYTE CONCENTRATION USING PHASE AND MAGNITUDE DETECTION OF A RADIATION TRANSFER FUNCTION; WIPO PCT Publication No. WO 01/30236 (corresponding to U.S. patent application Ser. No. 09/427,178), published May 3, 2001, titled SOLID-STATE NON-INVASIVE THERMAL CYCLING SPECTROMETER; U.S. Provisional Patent Application No. 60/336,404, filed Oct. 29, 2001, titled WINDOW ASSEMBLY; U.S. Provisional Patent Application No. 60/340,794, filed Dec. 11, 2001, titled REAGENT-LESS WHOLE-BLOOD GLUCOSE METER; U.S. Provisional Patent Application No. 60/340,435, filed Dec. 12, 2001, titled CONTROL SYSTEM FOR BLOOD CONSTITUENT MONITOR; U.S. Provisional Patent Application No. 60/340,654, filed Dec. 12, 2001, titled SYSTEM AND METHOD FOR CONDUCTING AND DETECTING INFRARED RADIATION; U.S. Provisional Patent Application No. 60/340,773, filed Dec. 11, 2001, titled METHOD FOR TRANSFORMING PHASE SPECTRA TO ABSORPTION SPECTRA; U.S. Provisional Patent Application No. 60/332,322, filed Nov. 21, 2001, titled METHOD FOR ADJUSTING SIGNAL VARIATION OF AN ELECTRONICALLY CONTROLLED INFRARED TRANSMISSIVE WINDOW; U.S. Provisional Patent Application No. 60/332,093, filed Nov. 21, 2001, titled METHOD FOR IMPROVING THE ACCURACY OF AN ALTERNATE SITE BLOOD GLUCOSE MEASUREMENT; U.S. Provisional Patent Application No. 60/332,125, filed Nov. 21, 2001, titled METHOD FOR ADJUSTING A BLOOD ANALYTE MEASUREMENT; U.S. Provisional Patent Application No. 60/341,435, filed Dec. 14, 2001, titled PATHLENGTH-INDEPENDENT METHODS FOR OPTICALLY DETERMINING MATERIAL COMPOSITION; U.S. Provisional Patent Application No. 60/339,120, filed Dec. 7, 2001, titled QUADRATURE DEMODULATION AND KALMAN FILTERING IN A BIOLOGICAL CONSTITUENT MONITOR; U.S. Provisional Patent Application No. 60/339,044, filed Nov. 12, 2001, titled FAST SIGNAL DEMODULATION WITH MODIFIED PHASE-LOCKED LOOP TECHNIQUES; U.S. Provisional Patent Application No. 60/336,294, filed Oct. 29, 2001, titled METHOD AND DEVICE FOR INCREASING ACCURACY OF BLOOD CONSTITUENT MEASUREMENT; U.S. Provisional Patent Application No. 60/338,992, filed Nov. 13, 2001, titled SITE SELECTION FOR DETERMINING ANALYTE CONCENTRATION IN LIVING TISSUE; and U.S. Provisional Patent Application No. 60/339,116, filed Nov. 7, 2001, titled METHOD AND APPARATUS FOR IMPROVING CLINICALLY SIGNIFICANT ACCURACY OF ANALYTE MEASUREMENTS. The entire disclosure of all of the above-mentioned patents, patent applications and publications are hereby incorporated by reference herein and made a part of this specification.

B. Whole-Blood Detection System

FIG. 13 is a schematic view of a reagentless whole-blood analyte detection system 200 (hereinafter “whole-blood system”) in a preferred configuration. The whole-blood system 200 may comprise a radiation source 220, a filter 230, a cuvette 240 that includes a sample cell 242, and a radiation detector 250. The whole-blood system 200 preferably also comprises a signal processor 260 and a display 270. Although a cuvette 240 is shown here, other sample elements, as described below, could also be used in the system 200. The whole-blood system 200 can also comprise a sample extractor 280, which can be used to access bodily fluid from an appendage, such as the finger 290, forearm, or any other suitable location.

As used herein, the terms “whole-blood analyte detection system” and “whole-blood system” are broad, synonymous terms and are used in their ordinary sense and refer, without limitation, to analyte detection devices which can determine the concentration of an analyte in a material sample by passing electromagnetic radiation through the sample and detecting the absorbance of the radiation by the sample. As used herein, the term “whole-blood” is a broad term and is used in its ordinary sense and refers, without limitation, to blood that has been withdrawn from a patient but that has not been otherwise processed, e.g., it has not been hemolysed, lyophilized, centrifuged, or separated in any other manner, after being removed from the patient. Whole-blood may contain amounts of other fluids, such as interstitial fluid or intracellular fluid, which may enter the sample during the withdrawal process or are naturally present in the blood. It should be understood, however, that the whole-blood system 200 disclosed herein is not limited to analysis of whole-blood, as the whole-blood system 10 may be employed to analyze other substances, such as saliva, urine, sweat, interstitial fluid, intracellular fluid, hemolysed, lyophilized, or centrifuged blood or any other organic or inorganic materials.

The whole-blood system 200 may comprise a near-patient testing system. As used herein, “near-patient testing system” is used in its ordinary sense and includes, without limitation, test systems that are configured to be used where the patient is rather than exclusively in a laboratory, e.g., systems that can be used at a patient's home, in a clinic, in a hospital, or even in a mobile environment. Users of near-patient testing systems can include patients, family members of patients, clinicians, nurses, or doctors. A “near-patient testing system” could also include a “point-of-care” system.

The whole-blood system 200 may in one embodiment be configured to be operated easily by the patient or user. As such, the system 200 is preferably a portable device. As used herein, “portable” is used in its ordinary sense and means, without limitation, that the system 200 can be easily transported by the patient and used where convenient. For example, the system 200 is advantageously small. In one preferred embodiment, the system 200 is small enough to fit into a purse or backpack. In another embodiment, the system 200 is small enough to fit into a pants pocket. In still another embodiment, the system 200 is small enough to be held in the palm of a hand of the user.

Some of the embodiments described herein employ a sample element to hold a material sample, such as a sample of biological fluid. As used herein, “sample element” is a broad term and is used in its ordinary sense and includes, without limitation, structures that have a sample cell and at least one sample cell wall, but more generally includes any of a number of structures that can hold, support or contain a material sample and that allow electromagnetic radiation to pass through a sample held, supported or contained thereby; e.g., a cuvette, test strip, etc. As used herein, the term “disposable” when applied to a component, such as a sample element, is a broad term and is used in its ordinary sense and means, without limitation, that the component in question is used a finite number of times and then discarded. Some disposable components are used only once and then discarded. Other disposable components are used more than once and then discarded.

The radiation source 220 of the whole-blood system 200 emits electromagnetic radiation in any of a number of spectral ranges, e.g., within infrared wavelengths; in the mid-infrared wavelengths; above about 0.8 μm; between about 5.0 μm and about 20.0 μm; and/or between about 5.25 μm and about 12.0 μm. However, in other embodiments the whole-blood system 200 may employ a radiation source 220 which emits in wavelengths found anywhere from the visible spectrum through the microwave spectrum, for example anywhere from about 0.4 μm to greater than about 100 μm. In still further embodiments the radiation source emits electromagnetic radiation in wavelengths between about 3.5 μm and about 14 μm, or between about 0.8 μm and about 2.5 μm, or between about 2.5 μm and about 20 μm, or between about 20 μm and about 100 μm, or between about 6.85 μm and about 10.10 μm.

The radiation emitted from the source 220 is in one embodiment modulated at a frequency between about one-half hertz and about one hundred hertz, in another embodiment between about 2.5 hertz and about 7.5 hertz, in still another embodiment at about 50 hertz, and in yet another embodiment at about 5 hertz. With a modulated radiation source, ambient light sources, such as a flickering fluorescent lamp, can be more easily identified and rejected when analyzing the radiation incident on the detector 250. One source that is suitable for this application is produced by ION OPTICS, INC. and sold under the part number NL5LNC.

The filter 230 permits electromagnetic radiation of selected wavelengths to pass through and impinge upon the cuvette/sample element 240. Preferably, the filter 230 permits radiation at least at about the following wavelengths to pass through to the cuvette/sample element: 3.9, 4.0 μm, 4.05 μm, 4.2 μm, 4.75, 4.95 μm, 5.25 μm, 6.12 μm, 7.4 μm, 8.0 μm, 8.45 μm, 9.25 μm, 9.5 μm, 9.65 μm, 10.4 μm, 12.2 μm. In another embodiment, the filter 230 permits radiation at least at about the following wavelengths to pass through to the cuvette/sample element: 5.25 μm, 6.12 μm, 6.8 μm, 8.03 μm, 8.45 μm, 9.25 μm, 9.65 μm, 10.4 μm, 12 μm. In still another embodiment, the filter 230 permits radiation at least at about the following wavelengths to pass through to the cuvette/sample element: 6.85 μm, 6.97 μm, 7.39 μm, 8.23 μm, 8.62 μm, 9.02 μm, 9.22 μm, 9.43 μm, 9.62 μm, and 10.10 μm. The sets of wavelengths recited above correspond to specific embodiments within the scope of this disclosure. Furthermore, other subsets of the foregoing sets or other combinations of wavelengths can be selected. Finally, other sets of wavelengths can be selected within the scope of this disclosure based on cost of production, development time, availability, and other factors relating to cost, manufacturability, and time to market of the filters used to generate the selected wavelengths, and/or to reduce the total number of filters needed.

In one embodiment, the filter 230 is capable of cycling its passband among a variety of narrow spectral bands or a variety of selected wavelengths. The filter 230 may thus comprise a solid-state tunable infrared filter, such as that available from ION OPTICS INC. The filter 230 could also be implemented as a filter wheel with a plurality of fixed-passband filters mounted on the wheel, generally perpendicular to the direction of the radiation emitted by the source 220. Rotation of the filter wheel alternately presents filters that pass radiation at wavelengths that vary in accordance with the filters as they pass through the field of view of the detector 250.

The detector 250 preferably comprises a 3 mm long by 3 mm wide pyroelectric detector. Suitable examples are produced by DIAS Angewandte Sensorik GmbH of Dresden, Germany, or by BAE Systems (such as its TGS model detector). The detector 250 could alternatively comprise a thermopile, a bolometer, a silicon microbolometer, a lead-salt focal plane array, or a mercury-cadmium-telluride (MCT) detector. Whichever structure is used as the detector 250, it is desirably configured to respond to the radiation incident upon its active surface 254 to produce electrical signals that correspond to the incident radiation.

In one embodiment, the sample element comprises a cuvette 240 which in turn comprises a sample cell 242 configured to hold a sample of tissue and/or fluid (such as whole-blood, blood components, interstitial fluid, intercellular fluid, saliva, urine, sweat and/or other organic or inorganic materials) from a patient within its sample cell. The cuvette 240 is installed in the whole-blood system 200 with the sample cell 242 located at least partially in the optical path 243 between the radiation source 220 and the detector 250. Thus, when radiation is emitted from the source 220 through the filter 230 and the sample cell 242 of the cuvette 240, the detector 250 detects the radiation signal strength at the wavelength(s) of interest. Based on this signal strength, the signal processor 260 determines the degree to which the sample in the cell 242 absorbs radiation at the detected wavelength(s). The concentration of the analyte of interest is then determined from the absorption data via any suitable spectroscopic technique.

As shown in FIG. 13, the whole-blood system 200 can also comprise a sample extractor 280. As used herein, the term “sample extractor” is a broad term and is used in its ordinary sense and refers, without limitation, to or any device which is suitable for drawing a sample of fluid from tissue, such as whole-blood or other bodily fluids through the skin of a patient. In various embodiments, the sample extractor may comprise a lance, laser lance, iontophoretic sampler, gas-jet, fluid-jet or particle-jet perforator, or any other suitable device.

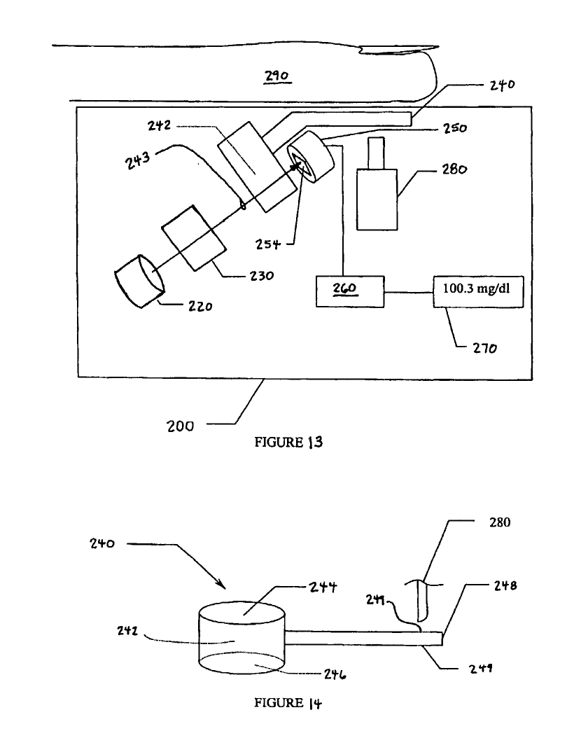

As shown in FIG. 13, the sample extractor 280 could form an opening in an appendage, such as the finger 290, to make whole-blood available to the cuvette 240. It should be understood that other appendages could be used to draw the sample, including but not limited to the forearm. With some embodiments of the sample extractor 280, the user forms a tiny hole or slice through the skin, through which flows a sample of bodily fluid such as whole-blood. Where the sample extractor 280 comprises a lance (see FIG. 14), the sample extractor 280 may comprise a sharp cutting implement made of metal or other rigid materials. One suitable laser lance is the Lasette Plus(t produced by Cell Robotics International, Inc. of Albuquerque, N. Mex. If a laser lance, iontophoretic sampler, gas-jet or fluid-jet perforator is used as the sample extractor 280, it could be incorporated into the whole-blood system 200 (see FIG. 13), or it could be a separate device.

Additional information on laser lances can be found in U.S. Pat. No. 5,908,416, issued Jun. 1, 1999, titled LASER DERMAL PERFORATOR, the entirety of which is hereby incorporated by reference herein and made a part of this specification. One suitable gas-jet, fluid-jet or particle-jet perforator is disclosed in U.S. Pat. No. 6,207,400, issued Mar. 27, 2001, titled NON- OR MINIMALLY INVASIVE MONITORING METHODS USING PARTICLE DELIVERY METHODS, the entirety of which is hereby incorporated by reference herein and made a part of this specification. One suitable iontophoretic sampler is disclosed in U.S. Pat. No. 6,298,254, issued Oct. 2, 2001, titled DEVICE FOR SAMPLING SUBSTANCES USING ALTERNATING POLARITY OF IONTOPHORETIC CURRENT, the entirety of which is hereby incorporated by reference herein and made a part of this specification.

FIG. 14 shows one embodiment of a sample element, in the form of a cuvette 240, in greater detail. The cuvette 240 further comprises a sample supply passage 248, a pierceable portion 249, a first window 244, and a second window 246, with the sample cell 242 extending between the windows 244, 246. In one embodiment, the cuvette 240 does not have a-second window 246. The first window 244 (or second window 246) is one form of a sample cell wall; in other embodiments of the sample elements and cuvettes disclosed herein, any sample cell wall may be used that at least partially contains, holds or supports a material sample, such as a biological fluid sample, and which is transmissive of at least some bands of electromagnetic radiation, and which may but need not be transmissive of electromagnetic radiation in the visible range. The pierceable portion 249 is an area of the sample supply passage 248 that can be pierced by suitable embodiments of the sample extractor 280. Suitable embodiments of the sample extractor 280 can pierce the portion 249 and the appendage 290 to create a wound in the appendage 290 and to provide an inlet for the blood or other fluid from the wound to enter the cuvette 240. (The sample extractor 280 is shown on the opposite side of the sample element in FIG. 14, as compared to FIG. 13, as it may pierce the portion 249 from either side.)

The windows 244, 246 are preferably optically transmissive in the range of electromagnetic radiation that is emitted by the source 220, or that is permitted to pass through the filter 230. In one embodiment, the material that makes up the windows 244, 246 is completely transmissive, i.e., it does not absorb any of the electromagnetic radiation from the source 220 and filter 230 that is incident upon it. In another embodiment, the material of the windows 244, 246 has some absorption in the electromagnetic range of interest, but its absorption is negligible. In yet another embodiment, the absorption of the material of the windows 244, 246 is not negligible, but it is known and stable for a relatively long period of time. In another embodiment, the absorption of the windows 244, 246 is stable for only a relatively short period of time, but the whole-blood system 200 is configured to observe the absorption of the material and eliminate it from the analyte measurement before the material properties can change measurably.

The windows 244, 246 are made of polypropylene in one embodiment. In another embodiment, the windows 244, 246 are made of polyethylene. Polyethylene and polypropylene are materials having particularly advantageous properties for handling and manufacturing, as is known in the art. Also, polypropylene can be arranged in a number of structures, e.g., isotactic, atactic and syndiotactic, which may enhance the flow characteristics of the sample in the sample element. Preferably the windows 244, 246 are made of durable and easily manufactureable materials, such as the above-mentioned polypropylene or polyethylene, or silicon or any other suitable material. The windows 244, 246 can be made of any suitable polymer, which can be isotactic, atactic or syndiotactic in structure.

The distance between the windows 244, 246 comprises an optical pathlength and can be between about 1 μm and about 100 μm. In one embodiment, the optical pathlength is between about 10 μm and about 40 μm, or between about 25 μm and about 60 μm, or between about 30 μm and about 50 μm. In still another embodiment, the optical pathlength is about 25 μm. The transverse size of each of the windows 244, 246 is preferably about equal to the size of the detector 250. In one embodiment, the windows are round with a diameter of about 3 mm. In this embodiment, where the optical pathlength is about 25 μm the volume of the sample cell 242 is about 0.177 μL. In one embodiment, the length of the sample supply passage 248 is about 6 mm, the height of the sample supply passage 248 is about 1 mm, and the thickness of the sample supply passage 248 is about equal to the thickness of the sample cell, e.g., 25 μm. The volume of the sample supply passage is about 0.150 μL. Thus, the total volume of the cuvette 240 in one embodiment is about 0.327 μL. Of course, the volume of the cuvette 240/sample cell 242/etc. can vary, depending on many variables, such as the size and sensitivity of the detectors 250, the intensity of the radiation emitted by the source 220, the expected flow properties of the sample, and whether flow enhancers (discussed below) are incorporated into the cuvette 240. The transport of fluid to the sample cell 242 is achieved preferably through capillary action, but may also be achieved through wicking, or a combination of wicking and capillary action.

FIGS. 15-17 depict another embodiment of a cuvette 305 that could be used in connection with the whole-blood system 200. The cuvette 305 comprises a sample cell 310, a sample supply passage 315, an air vent passage 320, and a vent 325. As best seen in FIGS. 16,16A and 17, the cuvette also comprises a first sample cell window 330 having an inner side 332, and a second sample cell window 335 having an inner side 337. As discussed above, the window(s) 330/335 in some embodiments also comprise sample cell wall(s). The cuvette 305 also comprises an opening 317 at the end of the sample supply passage 315 opposite the sample cell 310. The cuvette 305 is preferably about 1/4-⅛ inch wide and about 3/4inch long; however, other dimensions are possible while still achieving the advantages of the cuvette 305.

The sample cell 310 is defined between the inner side 332 of the first sample cell window 330 and the inner side 337 of the second sample cell window 335. The perpendicular distance T between the two inner sides 332, 337 comprises an optical pathlength that can be between about 1 μm and about 1.22 mm. The optical pathlength can alternatively be between about 1 μm and about 100 μm. The optical pathlength could still alternatively be about 80 μm, but is preferably between about 10 μm and about 50 μm. In another embodiment, the optical pathlength is about 25 μm. The windows 330, 335 are preferably formed from any of the materials discussed above as possessing sufficient radiation transmissivity. The thickness of each window is preferably as small as possible without overly weakening the sample cell 310 or cuvette 305.

Once a wound is made in the appendage 290, the opening 317 of the sample supply passage 315 of the cuvette 305 is placed in contact with the fluid that flows from the wound. In another embodiment, the sample is obtained without creating a wound, e.g. as is done with a saliva sample. In that case, the opening 317 of the sample supply passage 315 of the cuvette 305 is placed in contact with the fluid obtained without creating a wound. The fluid is then transported through the sample supply passage 315 and into the sample cell 310 via capillary action. The air vent passage 320 improves the capillary action by preventing the buildup of air pressure within the cuvette and allowing the blood to displace the air as the blood flows therein.

Other mechanisms may be employed to transport the sample to the sample cell 310. For example, wicking could be used by providing a wicking material in at least a portion of the sample supply passage 315. In another variation, wicking and capillary action could be used together to transport the sample to the sample cell 310. Membranes could also be positioned within the sample supply passage 315 to move the blood while at the same time filtering out components that might complicate the optical measurement performed by the whole-blood system 100.

FIGS. 16 and 16A depict one approach to constructing the cuvette 305. In this approach, the cuvette 305 comprises a first layer 350, a second layer 355, and a third layer 360. The second layer 355 is positioned between the first layer 350 and the third layer 360. The first layer 350 forms the first sample cell window 330 and the vent 325. As mentioned above, the vent 325 provides an escape for the air that is in the sample cell 310. While the vent 325 is shown on the first layer 350, it could also be positioned on the third layer 360, or could be a cutout in the second layer, and would then be located between the first layer 360 and the third layer 360 The third layer 360 forms the second sample cell window 335.

The second layer 355 may be formed entirely of an adhesive that joins the first and third layers 350, 360. In other embodiments, the second layer may be formed from similar materials as the first and third layers, or any other suitable material. The second layer 355 may also be formed as a carrier with an adhesive deposited on both sides thereof. The second layer 355 forms the sample supply passage 315, the air vent passage 320, and the sample cell 310. The thickness of the second layer 355 can be between about 1 μm and about 1.22 mm. This thickness can alternatively be between about 1 μm and about 100 μm. This thickness could alternatively be about 80 μm, but is preferably between about 10 μm and about 50 μm. In another embodiment, the second layer thickness is about 25 μm.

In other embodiments, the second layer 355 can be constructed as an adhesive film having a cutout portion to define the passages 315, 320, or as a cutout surrounded by adhesive.

Further information can be found in U.S. patent application No. 10/055,875, filed Jan. 22, 2002, titled REAGENT-LESS WHOLE-BLOOD GLUCOSE METER. The entire contents of this patent application are hereby incorporated by reference herein and made a part of this specification.

### II. Site Selection for Determining Analyte Concentration in Living Tissue

In this section are described methods and apparatus relating to the selection of a site at which are taken measurements of the concentration of an analyte within a material sample. In one embodiment, a restricted period commences after a subject eats, and the subject is permitted to take measurements only at “on-site” locations during the restricted period. When a restricted period is not in effect, the subject may take measurements at on-site or off-site locations.

A. Improvement of Measurement Accuracy

Glucose is present in blood vessels, cells, and interstitial fluid which is the fluid that bathes every cell in the body. Measurement of the concentration of blood glucose and/or other analytes/constituents, such as alcohol, at body sites other than the finger or fingertip (the “on-site” locations) is known as alternative site testing (AST). Alternative sites generally comprise any body location other than the finger or fingertip, including but not limited to the forearm, the upper forearm, the palm, the lower extremities, the abdomen, or any other location with sufficient blood circulation and healthy tissue.

In measuring the concentration of blood constituents such as glucose and/or alcohol, discrepancies are sometimes observed between measurements taken from blood resident at or drawn from on-site locations and measurements taken from blood resident at or drawn from alternative-site locations. A combination of parameters such as subject health, rate of constituent-concentration change, ambient temperature, measurement site temperature, choice of testing technique, etc., are believed to create the differences. In addition, blood flow to alternative sites is less efficient than to the fingertip. In some instances such a difference or a delay in equalization of glucose concentrations at on-site and alternative-site locations may be attributed to glucose absorbance by muscle tissue, in and around the alternative testing site. It can also be assumed that a variable supply of blood flow to the capillaries in the dermis of each test site defines the extent of any such difference or delay. These conditions may cause a degree of vasoconstriction and/or otherwise decrease the volume of blood flow to the forearm of other alternative sites as compared to the finger of fingertip. As a result, there is seen a decrease in the supply of the blood constituent of interest (such as glucose or alcohol) to the skin at alternative-site locations. If a difference arises between alternative-site and on-site glucose concentration readings in routine use, then it could lead to a problem for a patient with diabetes.

It has been found that augmenting local circulation at or near the measurement site will reduce or totally eliminate the difference or delay. Such augmentation is particularly effective for noninvasive measurements, such as noninvasive measurements taken with the noninvasive system 10 described above, because relatively small volumes of blood and interstitial fluid in the forearm (or at other alternative-site locations) can be involved in noninvasive measurements. However, it should be noted that this methodology is not limited to noninvasive measurements and may be used with invasive measurement techniques as well.

In accordance with one embodiment, there is provided a method and device for promoting circulation and increasing blood flow to a blood constituent measurement site. In short, an alternative site glucose monitor, such as a noninvasive, traditional, subcutaneous, continuous, and/or intermittent alternative site glucose monitor, can measure more accurately and counteract any lag with respect to on-site locations in blood glucose concentration and/or any other analyte of interest, by enhancing blood flow to the measurement site. The enhancement of blood flow can be achieved by a variety of local physical methods and/or pharmacological agents. Any such technique or device for increasing blood flow to the measuring site may be used, such as but not limited to one or more of the following or a combination thereof: applying a heater pad, rubbing the skin, squeezing the skin, tapping or thumping the skin, applying a topical agent such as, by way of example, BENGAY®, Mineral Ice® or Tiger Balm®, applying a vacuum to the measurement site, changing the air temperature around the measurement site, use of a vibration device, applying microwave/IR/ultrasonic or other energy. Any technique which enhances local circulation can be employed.

One presently preferred embodiment employs the application of heat to the skin site where the glucose/alcohol level (or other blood constituent) is to be tested. The heat interacts with glucose, at the test site, where it can be measured noninvasively. The heat also increases the local circulation which enhances the accuracy of the noninvasive readings by refreshing the glucose/alcohol content and flushing out potential contaminants and toxins that may cloud the readings.

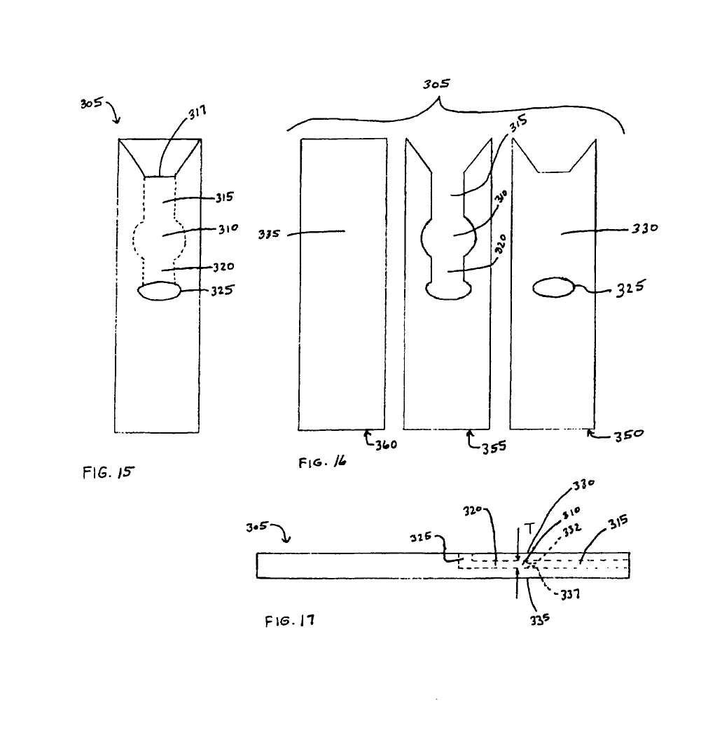

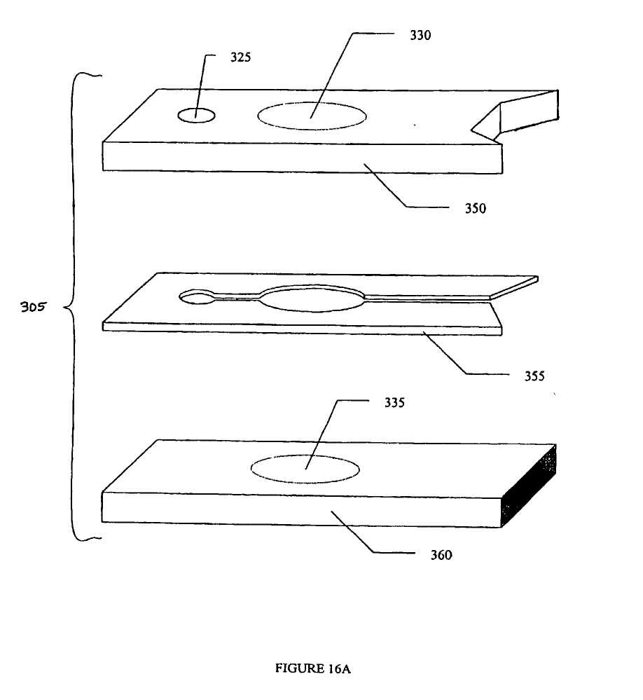

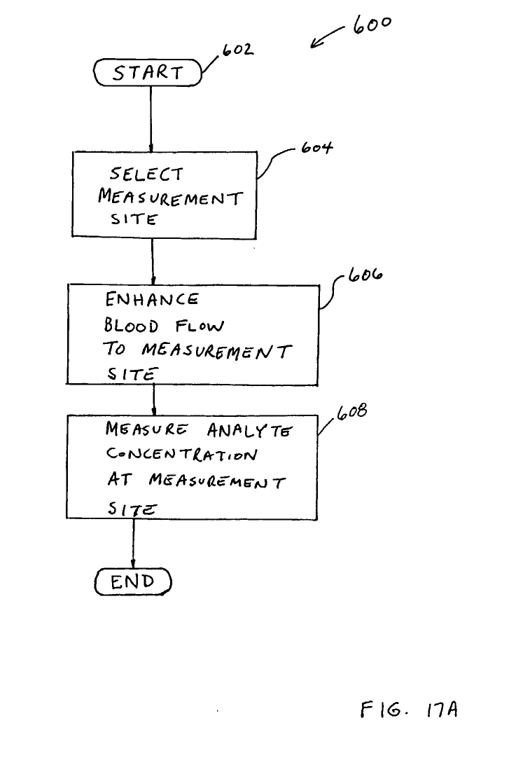

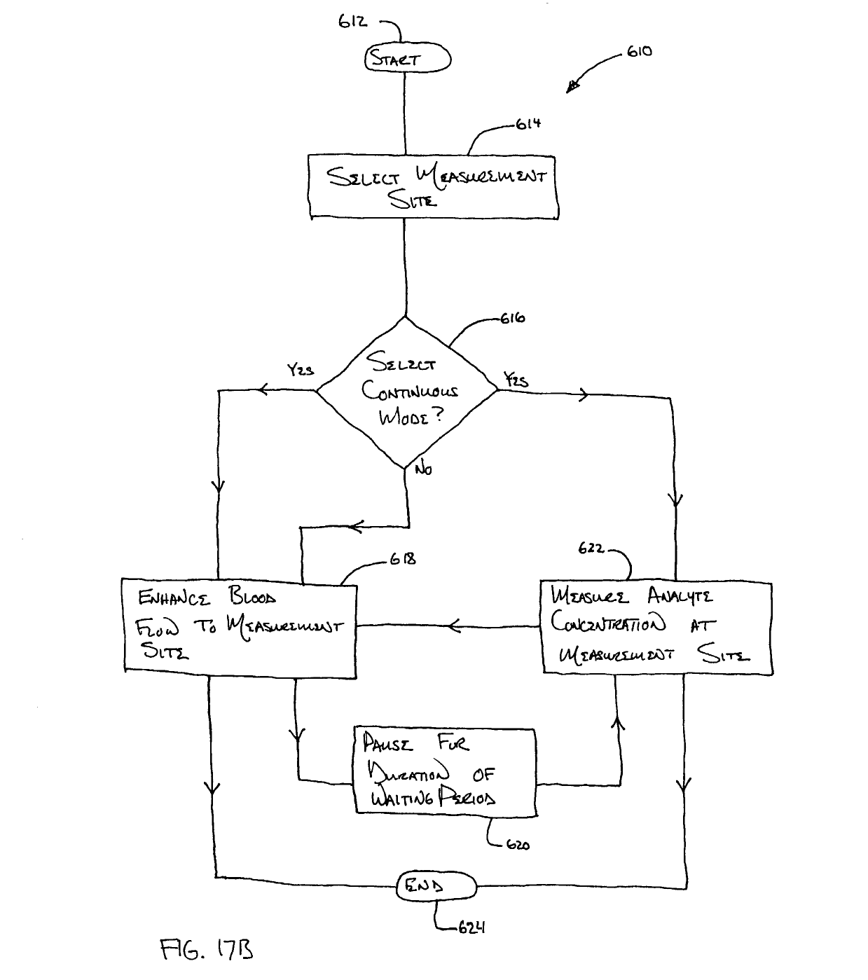

FIG. 17A is a flowchart depicting one embodiment of a method 600 for increasing the accuracy of an analyte concentration measurement. From a start state 602 the method 600 proceeds to a state 604 in which a measurement site is selected. In one embodiment, the measurement site comprises an alternative-site location; in another embodiment, the measurement site comprises an on-site location. As seen in state 606, the method 600 further comprises enhancing the degree of blood flow to the selected measurement site. This enhancement can be achieved by any of the techniques discussed herein. In state 608, the analyte concentration is measured at the measurement site. In one embodiment, the measurement is taken noninvasively; in another embodiment, the measurement is taken invasively. Noninvasive measurements may be taken with any suitable device, including but not limited to the noninvasive system 10 disclosed herein. Likewise, invasive measurements may be taken with any suitable device, including but not limited to the whole-blood system 200 disclosed herein. As further alternatives (or in addition to these techniques), the measurement can be taken subcutaneously (i.e., with an implantable or semi-implantable measurement device, continuously, or intermittently.

The method 600 may be employed in taking measurements of the concentration of a wide variety of analytes such as glucose, alcohol, or any other analyte mentioned above, within a patient's blood, interstitial fluid or intercellular fluid, or any combination thereof. Furthermore, the method 600 may be employed with any one or combination of the alternative-site locations or on-site locations disclosed above.

In one embodiment, a heat source, such as any suitable or commercially available heating pad, hair dryer, heat gun, hot-water bottle, or any other heat source, is applied to the measurement site to enhance local blood flow. Alternatively or additionally, the skin may be rubbed, tapped and/or squeezed, either manually or with any suitable or commercially available hand-held massage device. Vibration or a vacuum may be applied to the measurement site by employing any suitable or commercially available vibration massager or vacuum device. Other energy, such as microwave, infrared, or ultrasonic energy may be applied to the measurement site by any suitable or commercially available energy source. A vasodilating topical agent or irritant such as alprostadil (available in topical form under the trade name ALISTA from Vivus, Inc. of Mountain View, Calif.) may be applied to the measurement site, or a vasodilating pharmacological agent, drug, or chemical can be ingested to induce local or systemic vasodilation.

Any of the physical blood flow enhancement techniques discussed above may be employed for any suitable duration to improve the accuracy of a subsequent analyte concentration measurement. In a preferred embodiment, the enhancement technique is applied for a period ranging between 5 and 20 seconds; in other embodiments, the enhancement technique may be applied for 15 seconds, 30 seconds, one minute, 2 minutes, 5 minutes, 10 minutes, 15 minutes or more.

In one embodiment, a suitable waiting period is interposed between blood flow enhancement and analyte concentration measurement. The waiting period will vary with the particular blood flow enhancement technique(s) employed. For example, when a physical method is utilized, the waiting period may last from about 10-15 seconds to about 10-15 minutes. When a vasodilating drug is taken, the waiting period may last from about 30 minutes to about 90 minutes or more.

FIG. 17B is a flowchart illustrating another embodiment of a method 610 for increasing the accuracy of an analyte concentration measurement. From a start state 612, the method 610 proceeds to a state 614 in which a measurement site is selected. In one embodiment, the measurement site comprises an alternative-site location; in another embodiment, the measurement site comprises an on-site location. Once the measurement site is selected, the method 610 proceeds to state 616 in which a continuous mode may or may not be selected. When the continuous mode is not selected the method 610 proceeds to state 618 in which the degree of blood flow to the measurement site is enhanced. This enhancement can be achieved by any of the techniques discussed herein. As seen in state 620, after blood flow to the measurement site has been enhanced, the method 610 pauses during a waiting period before proceeding to state 622. The length of the waiting period will vary depending on the particular blood flow enhancement techniques(s) employed. For instance, when a tapping or thumping of the skin is utilized, the waiting period may be about 10 seconds. In one embodiment, wherein tapping of the skin is employed, the waiting period may range between 5 seconds and 20 seconds. In another embodiment, the waiting period may be set to essentially 0 seconds, thereby substantially eliminating the waiting period from the method 610.

In state 622, the analyte concentration is measured at the measurement site. In one embodiment, the measurement is taken noninvasively; in another embodiment, the measurement is taken invasively. Noninvasive measurements may be taken with any suitable device, including but not limited to the noninvasive system 10 disclosed herein. Likewise, invasive measurements may be taken with any suitable device, including but not limited to the whole-blood system 200 disclosed herein. Once the analyte concentration is measured at the measurement site, the method 610 returns to state 618 in which blood flow to the measurement site is enhanced again. In one embodiment, the method 610 periodically cycles through the states 618, 620 and 622. Once a number of cycles suitable for determining the analyte concentration have been performed, the method 610 proceeds to from state 622 to an end state 620.

Referring again to state 616, when the continuous mode is selected, the method 610 advances to both states 618 and 622 at the same time. Thus, in the continuous mode blood flow enhancement and analyte concentration measurement are advantageously performed simultaneously at the measurement site. As will be appreciated by those skilled in the art, the above-discussed embodiment of the noninvasive system 10 comprising an agitator may perform one or more of the above-discussed enhancement techniques while simultaneously and continuously measuring the analyte concentration at the selected measurement site.

It is contemplated that the method 610 is particularly suitable for use with noninvasive and invasive measurement devices that are capable of intermittently and/or simultaneously performing the above-discussed blood flow enhancement techniques and measuring analyte concentration at the measurement site. For instance, in one embodiment the noninvasive system 10 may comprise an agitator (such as but not limited to a vibrator or vibrating plate, reciprocating/massaging projections, ultrasonic transducer, vacuum nozzle, IR emitter, heat source, hot-air blower, or any other suitable structure) capable of performing one or more of the above-discussed blood flow enhancement techniques. In this embodiment, the noninvasive system 10 may alternate between an agitation period, during which one or more enhancement techniques are applied to the measurement site, and a measuring period during which the analyte concentration is measured. The agitation period will vary with the particular blood flow enhancement technique(s) employed. In one embodiment, the agitation period may be about 10 seconds, and the measurement period may be about 3 minutes. In another embodiment, the agitation period may range between about 5 seconds and about 20 seconds while the measurement period is not greater than 3 minutes. In still another embodiment, the lengths of the agitation and measurement periods may increase or decrease over time. Still, in another embodiment one or more blood flow enhancement techniques and analyte measurement may advantageously be performed simultaneously at the measurement site.

As will be appreciated by those skilled in the art, enhancement techniques for agitating the skin at the measurement site may introduce “noise” into, or otherwise affect, concurrent analyte concentration measurements at the selected site. This noise can arise physically at the detectors 28 or can manifest as post-detector electronic noise. For example, if tapping or thumping of the skin is utilized to enhance local blood flow, physical noise can arise due to vibration and/or movement of the skin. Likewise, similar effects can arise due to movement, shock or vibration of the noninvasive system 10 and/or relative movement between the noninvasive system 10 and the measurement site. In one embodiment, the noninvasive system 10 may comprise non-microphonic detectors 28 order to mitigate physical noise. In other embodiments, the noninvasive system 10 may comprise electronic means for reducing or substantially eliminating electronic noise effects. Still, if a heat source is applied to the measurement site to enhance local blood flow, “temperature noise” can arise due to the additional heat supplied by the heat source. As with other forms of noise, temperature noise can affect the outcome of analyte concentration measurements. It is contemplated that depending on the type of agitator utilized with the noninvasive system 10, analyte concentration measurements account for the presence of noise arising due to a wide variety of phenomena, including but not limited to, electronic effects, temperature changes, movement, shock and vibration of the noninvasive system 10 and/or the measurement site, relative movement between the noninvasive system 10 and the measurement site, and the like.

B. Site Selection

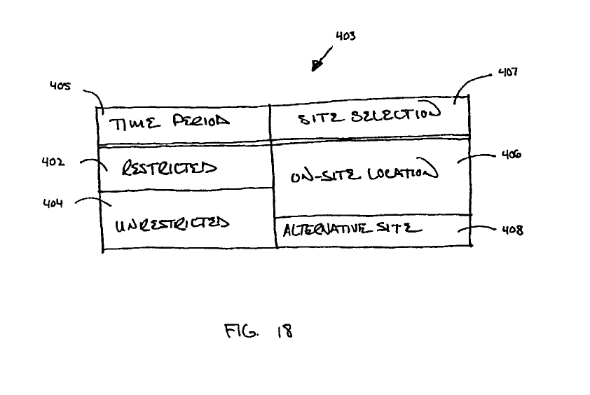

FIG. 18 is a table 403 which illustrates a relationship between time periods 405 and site selections 407 on the subject's body whereat analyte measurements may be taken. The time periods 405 comprise a restricted time period 402 and an unrestricted time period 404. The site selections 407 comprise an on-site location 406 and an alternative site 408. As shown, the restricted time period 402 corresponds only with the on-site location 406. The restricted time period 402 commences immediately after the subject eats. As used herein, the term “eat” is a broad term and is used in its ordinary sense and refers, without limitation, to any ingestion of nourishment, solid or liquid, orally, intravenously, or otherwise, as well as any ingestion of any drug or agent, orally, intravenously, or otherwise, which tends to raise or lower the concentration of the analyte(s) of interest in a subject's blood, bodily fluids, tissue, etc. In other embodiments, the restricted time period 402 commences immediately after any event which causes rapid changes in blood glucose concentration. During the restricted time period 402, the subject is restricted to taking analyte measurements at the on-site location 406. In a preferred embodiment, the restricted time period 402 is about 2.0 hours. In another embodiment, the restricted time period 402 may range between about 0.5 hours and about 3 hours. In still another embodiment, the restricted time period 402 may range between about 1.0 hours and about 2.0 hours. In yet another embodiment, the restricted time period 402 may range between about 1.5 hours and about 2.0 hours.

As further illustrated in FIG. 18, the unrestricted time period 404 commences after the restricted time period 402 has elapsed. (It is contemplated that, should the subject eat during a restricted period, the restricted period commences anew and lasts for the designated time period.) The unrestricted time period 404 corresponds with both the onsite location 406 and the alternative site 408. The unrestricted time period 404 provides a choice of locations on the subject's body whereat analyte measurements may be taken. During the unrestricted time period 404, the subject may take analyte measurements either at the on-site location 406 or at the alternative site 408 such as, for example, the forearm. As mentioned above, it is to be understood that “alternative site” refers to any location on the body other than the on-site location 406.

In the method depicted in FIG. 18, analyte concentration measurements may be taken with any suitable method, including but not limited to invasive and noninvasive methods. Noninvasive measurements may be taken with any suitable device, including but not limited to the noninvasive system 10 disclosed herein. Invasive measurements may be taken with any suitable device, including but not limited to the whole-blood system 200 disclosed herein. The method may be employed in measuring the concentration of any analyte disclosed herein, including but not limited to glucose and/or alcohol, or any combination of analytes.

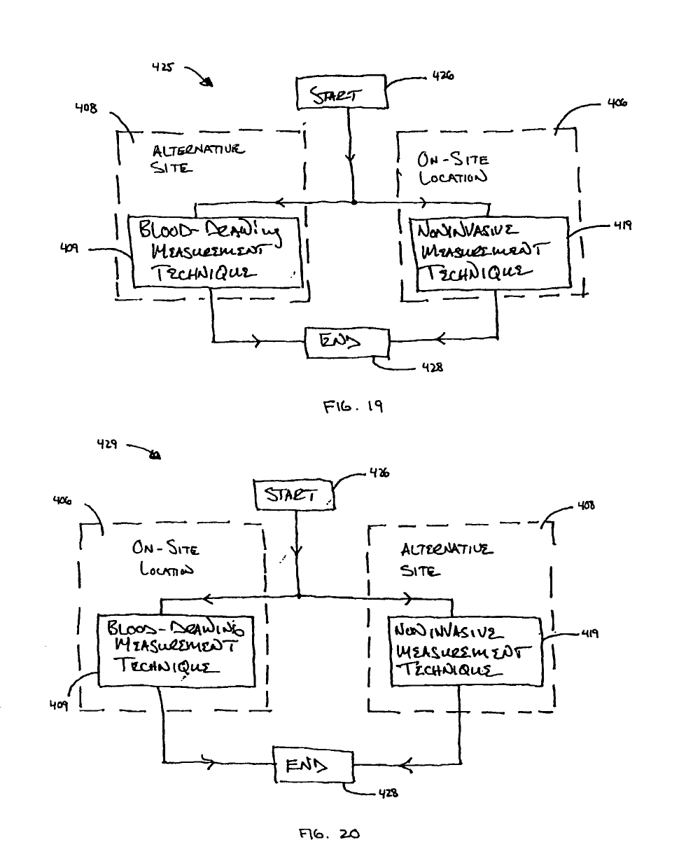

FIG. 19 illustrates one embodiment of an analysis procedure 425 whereby analyte concentration measurements are taken at a suitable site on the subject's body based on the amount of elapsed time after the subject has eaten. As shown, the analysis procedure 425 initiates with a start state 426 wherein the subject determines the elapsed time since having last eaten, and thus determines whether or not the current time is within the restricted time period 402. If the elapsed time is within the restricted time period 402, then analyte concentration measurements must be performed at the on-site location 406. However, if the elapsed time is within the unrestricted time period 404, then analyte concentration measurements may be taken at either the on-site location 406 or the alternative site 408.

Once the subject determines whether or not analyte concentration measurements must be taken at the on-site location 406, the subject can then decide between using invasive or noninvasive measurement techniques. In the embodiment illustrated in FIG. 19, a noninvasive process 419 is used at the on-site location 406, and a blood drawing process 409 is used at the alternative site 408. The noninvasive process 419 comprises using a noninvasive monitor to capture and determine the analyte concentration data within the subject's tissue. The noninvasive monitor includes, for example, a monitor of the type which detects infrared energy emitted and/or reflected by the subject's tissue to determine the analyte concentration based on the amount of infrared energy absorbed by the analyte. In one embodiment, the noninvasive monitor comprises the above-discussed noninvasive system 10 (FIG. 1). The blood drawing process 409, performed at only the alternative site 408, comprises taking a sample of blood, interstitial fluid, intracellular fluid, or any combination thereof, from the subject to determine the analyte concentration within the subject's tissue. It is contemplated that the blood and/or fluid sample is analyzed with any of various known and commercially available optical or electrochemical devices, test strips, etc., designed for analysis of drawn blood or fluid samples, or any other suitable apparatus, including but not limited to the whole-blood system 200 disclosed herein. After the analyte concentration within the subject's tissue is determined, the analysis procedure 425 ends at state 428. (It should be noted that “blood-drawing” as used herein or in the Figures refers to any invasive measurement technique.)

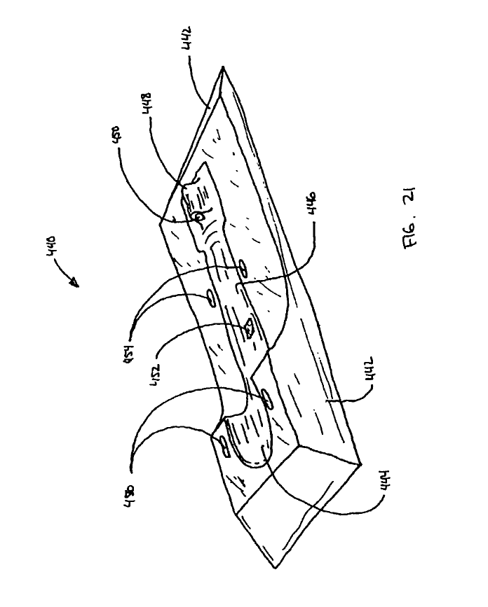

FIG. 20 illustrates another embodiment of an analysis procedure 429 whereby analyte concentration measurements are taken at a suitable site on the subject's body based on the amount of elapsed time after the subject has eaten. The procedure 429 illustrated in FIG. 20 is substantially similar to the procedure 425 shown in FIG. 19, with the exception that in the procedure 429 the blood drawing process 409 is performed at the on-site location 406 and the noninvasive procedure 419 is performed at the alternative site 408. As shown in FIG. 20, the procedure 429 begins with the state 426 wherein the subject determines the amount of elapsed time since having eaten. If the elapsed time is found to be within the restricted time period 402, then analyte concentration measurements must be performed at only the on-site location 406. If the elapsed time is within the unrestricted time period 404, however, then analyte concentration measurements may be taken either at the on-site location 406 or at the alternative site 408.

After the subject determines whether or not analyte concentration measurements must be taken at the on-site location 406, the subject then decides between using the noninvasive process 419 or the blood drawing process 409. In the embodiment illustrated in FIG. 20, the blood drawing process 409 is used at the on-site location 406, and the noninvasive process 419 is used at the alternative site 408. Once the analyte concentration within the subject's tissue is determined, the procedure 429 then ends at the state 428.

Upon considering the analysis procedures 425, 429 in view of FIG. 18, a person of ordinary skill in the art will recognize that the subject may choose between the procedures 425, 429 so as to use one type of measurement technique only at the on-site location 406, during the restricted time period 402, while using the other technique only for the alternative site 408, during the unrestricted time period 404. Still, the subject may use one of the techniques only at the on-site location 406, during the restricted time period 402, and during the unrestricted time period 404 the subject may use the other technique for the alternative site 408 and/or the on-site location 406. As an alternative the subject may use either technique at any time of the day and/or at either the on-site location 406 or the alternative site 408.

It should be further noted that any of the methods disclosed above in connection with FIGS. 17A, 18, 19 and 20 can be employed in connection with an implantable blood-constituent sensor, such as an implantable blood-glucose sensor. Any suitable implantable sensor, including implantable optical sensors, may be employed, including but not limited to those disclosed in U.S. Pat. No. 6,122,536, issued Sep. 19, 2000, titled IMPLANTABLE SENSOR AND SYSTEM FOR MEASUREMENT AND CONTROL OF BLOOD CONSTITUENT LEVELS; and U.S. Pat. No. 6,049,727, issued Apr. 11, 2000, titled IMPLANTABLE SENSOR AND SYSTEM FOR MEASUREMENT AND CONTROL OF BLOOD CONSTITUENT LEVELS. The entire contents of the above-noted patents are hereby incorporated by reference herein and made a part of this specification.

C. Stabilization Devices

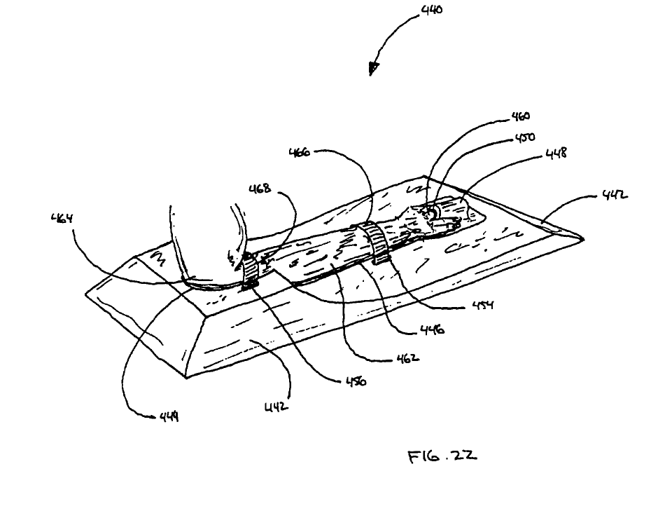

FIG. 21 is a perspective view of one embodiment of a mechanical stabilization device 440 which can be employed to immobilize the subject's finger and/or hand when exposing it to an noninvasive monitor for on-site and/or alternative-site measurements. (It should be noted that the devices discussed in this section are presented in an exemplary use with a noninvasive monitor, but the devices may also be used with noninvasive monitors where suitable.) In the embodiment illustrated in FIG. 21, the mechanical stabilization device 440 comprises a base 442, an elbow channel 444, a forearm channel 446, a finger restraint 448, and a finger hole 450. The elbow and forearm channels 444, 446, as well as the finger restraint 448, are formed so as to conform to the anatomical shape of the subject's arm and fingers. The base 442 further comprises a pair of forearm restraining holes 454 and a pair of elbow restraining holes 456. The forearm restraining holes 454 and the elbow restraining holes 456 facilitate stabilizing the subject's arm within the forearm channel 446 and the elbow channel 444, respectively, such that relative movement between the arm and the base 446 is substantially minimized.

As shown, the forearm channel 446 includes a primary window 452. The primary window 452 is configured to interface with a window of the noninvasive monitor, such as the noninvasive system 10 or the window or the thermal mass window of the apparatus taught in the above-mentioned U.S. Pat. No. 6,198,949. The primary window 452 facilitates capturing analyte concentration data within tissue at the alternative site 408 (i.e., the subject's forearm). It is contemplated that a secondary window (not shown) is included within the finger hole 450. The secondary window is substantially similar to the primary window 452 with the exception that the secondary window is smaller than the primary window 452. More specifically, the secondary window is smaller than the primary window 452 and is configured to fit within the finger hole 450. As with the primary window 452, the secondary window interfaces with the noninvasive monitor, and facilitates the capture of analyte concentration data within tissue at the on-site location 406 (i.e., the subject's finger).

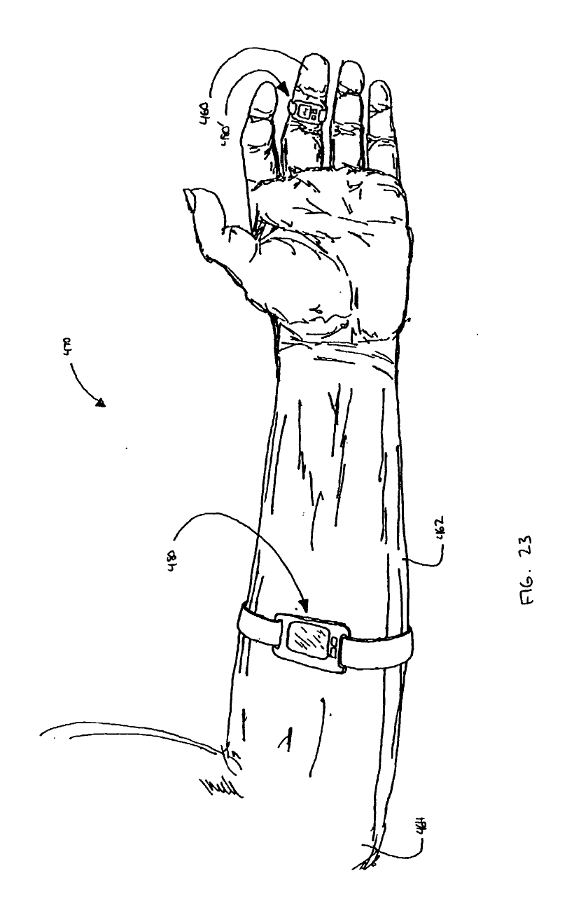

FIG. 22 illustrates the mechanical stabilization device 440 in an exemplifying use environment wherein the device 440 is stabilizing the subject's arm for determination of analyte concentration within the subject's tissue. As shown, a finger 460 is inserted into the finger hole 450 while the other fingers rest on either side of the finger restraint 448. A forearm 462 is laid onto the forearm channel 446 and an elbow 464 is placed within the elbow channel 444. A fastening strap 466 is passed over the forearm 462 and through the forearm fastening holes 454 and then tightened to prevent relative movement between the forearm 462 and the base 442. Similarly, a fastening strap 468 is passed over a proximal portion of the forearm 462 and through the fastening holes 456 and then tightened to prevent relative movement between the elbow 464 and the base 442. Additionally, it is contemplated that the finger hole 450 has a diameter which may be increased and decreased so as to tighten around and release the subject's finger 460. This stabilizes the finger 460 so as to prevent relative motion of the finger 460 within the hole 450. Tightening of the finger hole 450 serves the additional purpose of pressing the finger 460 against the above-mentioned secondary window within the finger hole 450, thereby placing the finger 460 in thermal contact with the secondary window. With the forearm 462, the elbow 464, and the finger 460 sufficiently stabilized, the subject initiates the determination of analyte concentration within the subject's tissue.

As will be apparent to those of ordinary skill in the art, the base 442 of the device 440 must be adjustable in order to conform to the sizes and shapes of the arms and fingers of a variety of subjects. It is contemplated that the finger restraint 448 is movable distally and proximally relative to the forearm channel 446 to accommodate various forearms 462 and fingers 460 having different lengths. It is further contemplated that the diameter of the finger hole 450 may be increased and decreased so as to stabilize a variety of fingers having different sizes.

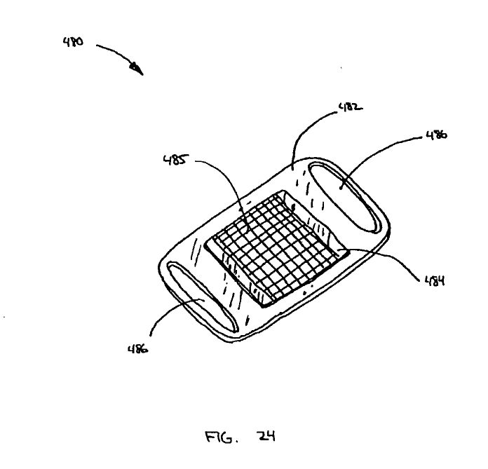

FIG. 23 is a perspective view of another embodiment of a mechanical stabilization device 470, illustrated in an exemplifying use environment. As shown, the stabilization device 470 comprises a first wearable window 480 fastened to the forearm 462 and a second, somewhat smaller wearable window 480′ which is fastened to the finger 460. It is contemplated that the wearable windows 480, 480′ are to be used in conjunction with a noninvasive monitor such as, but not necessarily limited to, the noninvasive system 10 as well as the apparatus taught in the above-mentioned U.S. Pat. No. 6,198,949. This patent discloses a noninvasive thermal gradient spectrometer comprising a window and a thermal mass window, wherein the window forms an interface between a thermal mass window and a subject's skin. It is contemplated that the wearable windows 480, 480′ each effectively takes the place of the window of the thermal gradient spectrometer, and thus forms the interface between the thermal mass window and the subject's skin. It is further contemplated that both the wearable windows 480, 480′ may advantageously be used with the same thermal gradient spectrometer. While the wearable window 480 is sized for an optimal interface with the thermal mass window, the wearable window 480′ may be used with an adaptive member (not shown). The adaptive member facilitates coupling the smaller wearable window 480′ with the thermal mass window of the thermal gradient spectrometer. Furthermore, it is contemplated that the wearable windows 480, 480′ may be used in conjunction with the noninvasive system 10 in accordance with the methodology taught in the above-mentioned U.S. Pat. No. 6,161,028.

As illustrated in FIG. 23, the wearable windows 480, 480′ are tightly fastened to the forearm 462 and the finger 460, respectively, such that the wearable windows 480, 480′ are placed into intimate thermal contact with the subject's skin. It is contemplated that each of the wearable windows 480, 480′ is electrically connected to a power supply (not shown) which resides on the noninvasive system 10, or otherwise externally thereto. In another embodiment, the wearable window 480 is connected to a first power supply and the wearable window 480′ is connected to a second power supply. In still another embodiment, a power cable may be extended from the first wearable window 480 to the second wearable window 480′. It is contemplated that the power cable places the wearable windows 480, 480′ in direct electrical communication whereby the second wearable window 480′ receives electric power when the first wearable window 480 is connected to the power supply. As will be appreciated by those skilled in the art, a wide variety of techniques, materials and configurations may advantageously be used for supplying electric power to the wearable windows 480, 480′.

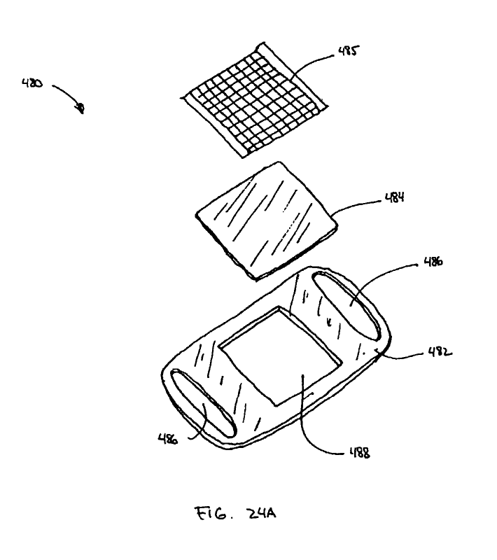

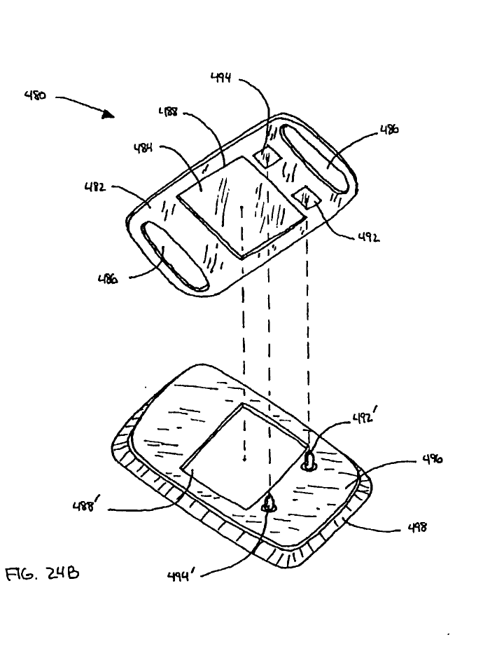

FIG. 24 is a perspective view of one embodiment of the wearable window 480. The illustrated embodiment of FIG. 24 is substantially similar to an apparatus described in Assignee's copending provisional application, entitled DEVICE FOR CAPTURING THERMAL SPECTRA FROM TISSUE, Serial No. 60/310,898, filed Jul. 17, 2001, the entirety of which is hereby incorporated by reference. In the embodiment illustrated in FIG. 24, the wearable window 480 comprises a window holder 482, a substrate 484, a heating element 485, and openings 486 to facilitate fastening the wearable window 480 to the subject (see FIG. 23). FIG. 24A is an exploded view of the wearable window 480, which illustrates the several elements comprising the wearable window 480. As can be seen most clearly in FIG. 24A, the window holder 482 serves as a foundation upon which the several elements comprising the wearable window 480 may advantageously be affixed. Furthermore, the window holder 482 serves to facilitate attaching the wearable window 480 to the subject's skin such that the wearable window 480 assumes intimate thermal contact therewith (see FIG. 23). The window holder 482 may be formed of injection-molded plastic or other similar material such that the several elements comprising the wearable window 480 may be affixed to the window holder 482 with minimal movement arising therebetween. It is further contemplated that the material comprising the window holder 482 may be such that condensation formed thereon when the window holder 482 is exposed to cooler temperatures (below the dew point) is substantially minimized.

As illustrated in FIG. 24A, the window holder 482 further comprises an aperture 488. The aperture 488 allows unimpeded transmission of thermal spectra through the window holder 482 to and from the subject's skin. Although in the embodiment of FIG. 24A the aperture 488 has a rectangular cross-sectional shape, it is contemplated that the aperture 488 may have other cross-sectional shapes, such as, by way of example, square, circular, diamond, elliptical, and ovoid. It is further contemplated that different cross-sectional shapes may advantageously be combined, thereby forming additional cross-sectional shapes.

Disposed upon or within the aperture 488 of the window holder 482 is the substrate 484. The substrate 484 preferably has a length and a width that are somewhat greater than the length and width of the aperture 488, thereby facilitating fastening of the substrate 484 to the window holder 482. In one embodiment, the substrate 484 is permanently affixed to the window holder 482. In another embodiment, the substrate 484 may be removably attached to the window holder 482. In still another embodiment, the substrate 484 may comprise a disposable member which is attachable to and detachable from the window holder 482.

The substrate 484 preferably is made of a material having a high thermal conductivity, such as polycrystalline float zone silicon, diamond, CVD diamond, or other similar material, such that the substrate 484 is substantially transparent to thermal spectra. In addition, the substrate 484 may have a thickness sized such that thermal spectra are substantially unimpeded as they transfer through the substrate 484. In the illustrated embodiment of FIGS. 24 and 24A, the substrate 484 preferably has a thickness of about 0.25 millimeters. It will be appreciated by those of ordinary skill in the art, however, that the material comprising the substrate 484, as well as the dimensions thereof, may advantageously be changed.

Disposed upon the substrate 484 is the heating element 485, which is substantially similar to the heater layer 34 discussed with reference to FIG. 1. The heating element 485 transfers heat to the skin of the subject, and thus gives rise to the heating component of the aforementioned intermittent heating and cooling of the subject's skin. The heating element 485 preferably comprises a first adhesion layer of gold or platinum (i.e., the above-discussed “gold layer”) deposited over an alloy layer which is applied to the substrate 484. The alloy layer comprises a material suitable for implementation of the heating element 485, such as 10/90 titanium/tungsten, titanium/platinum, nickel/chromium, or other similar material. As discussed with reference to FIG. 1, the gold layer preferably has a thickness of about 4000 Å, and the alloy layer preferably has a thickness ranging between about 300 Å and about 500 Å. The gold layer and/or the alloy layer may be deposited onto the substrate 484 by chemical deposition including, but not necessarily limited to, vapor deposition, liquid deposition, plating, laminating, casting, sintering, or other forming or deposition methodologies well known to those or ordinary skill in the art. Further details regarding the manufacture and/or fabrication of the heating element 485 are discussed herein with reference to FIG. 1 and in the above-mentioned U.S. Pat. No. 6,198,949.

As will be appreciated by a person skilled in the art, in an alternative embodiment the heating element 485 may be omitted from the wearable window 480. It is contemplated that with this embodiment, the wearable window 480 comprises the window holder 482 and the substrate 484, while an element similar in function to the heating element 485 is provided by the noninvasive system 10 or other optical measurement system with which the wearable window 480 is intended to be used. It is further contemplated that this embodiment of the wearable window 480 would be particularly useful with optical measurement systems wherein a heat source has been omitted. In such instances, heating of the subject's skin is accomplished by allowing the skin to warm up naturally to the ambient temperature of the surrounding environment.

FIG. 24B illustrates one embodiment of an electrical connection established between the wearable window 480 and an optical measurement system 498, whereby electrical power may advantageously be supplied to the heating element 485. In the embodiment illustrated in FIG. 24B, the wearable window 480 comprises a first contact 492 and a second contact 494. The contacts 492, 494 are made of an electrically conducting material, such as gold, silver, copper, steel, brass, or other similar material, which is molded into the material comprising the window holder 482. It is contemplated that the contacts 492, 494 are in electrical communication with the heating element 485 (see FIGS. 24 and 24A).

As shown, the first contact 492 directly corresponds with a first pin 492′ protruding from an interface surface 496 of the optical measurement system 498. Similarly, the second contact 494 directly corresponds with a second pin 494′ protruding from the interface surface 496. The pins 492′, 494′ are slidably retained within sockets (not shown) and are spring biased such that they are in a neutral, protruded state relative to the interface surface 496. When the wearable window 480 is pressed against the interface surface 496, the pins 492′, 494′ are pushed into the sockets while being urged against the contacts 492, 494. It is contemplated that the pins 492′, 494′ are made of an electrically conducting material, such as gold, silver, copper, steel, brass, or other similar material, and are in electrical communication with a switched power supply (not shown) which resides on the optical measurement system 498 or externally thereto.

The interface surface 496 may be made of rubber or other semi-compliant material which grips the wearable window 480, thereby preventing relative motion between the wearable window 480 and the optical measurement system 498. The interface surface 496 includes an aperture 488′ which directly corresponds with the aperture 488 of the wearable window 480. The aperture 488′ allows thermal spectra unimpeded passage between the wearable window 480 and the optical measurement system 498.

As shown in the embodiment of FIG. 24B, the interface surface 496 has a thickness which provides a thin layer of airspace between a window (not shown) of the optical measurement system 498 and the substrate 484. In another embodiment, however, the substrate 484 may have a thickness such that when the wearable window 480 is pressed against the interface surface 496, a portion of the substrate 484 extends through the aperture 488′ and comes into thermal contact with the window of the optical measurement system 498.

In operation, the wearable window 480 is fastened to the skin of the subject and then is pressed against the interface surface 496 such that the apertures 488, 488′ are centered and aligned, and electrical communication is respectively established between the pins 492′, 494′ and the contacts 492, 494. As the wearable window 480 is further pressed onto the interface surface 496, the pins 492′, 494′ and the contacts 492, 494 remain in electrical communication as the pins are pushed into their respective sockets.

Once the wearable window 480 is sufficiently pressed against the interface surface 496, the heating element 485 is placed into electrical communication with the above-mentioned switched power supply (not shown), whereby intermittent heating is applied to the skin. The optical measurement system 498 is placed in thermal contact with the substrate 484 such that the substrate 484 and the heating element 485 together form an interface between the optical measurement system 498 and the subject's skin.

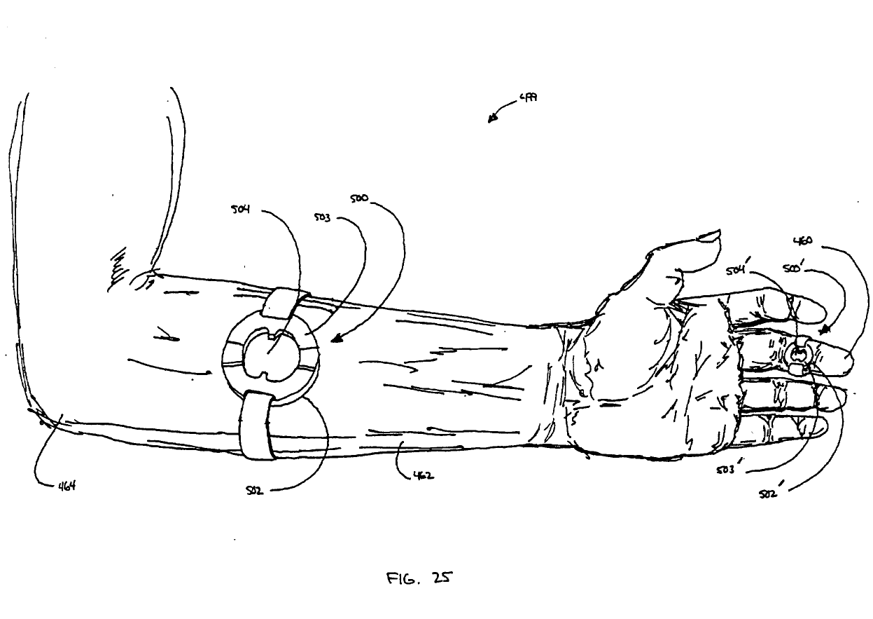

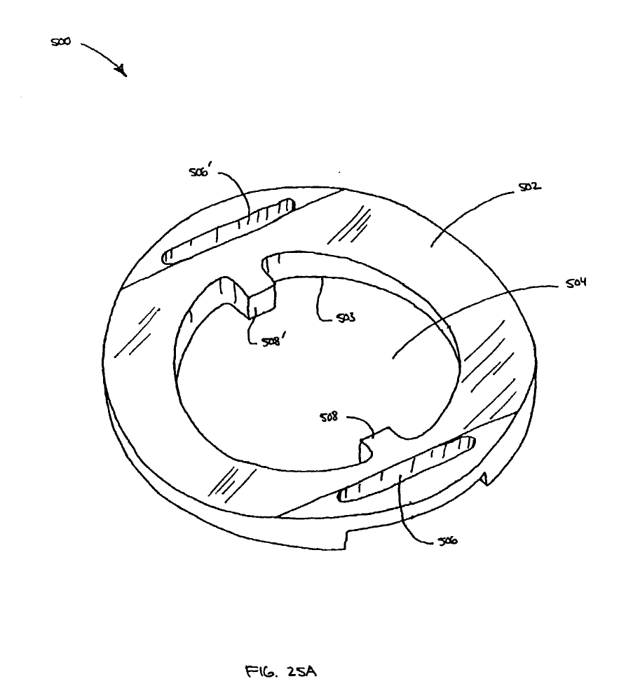

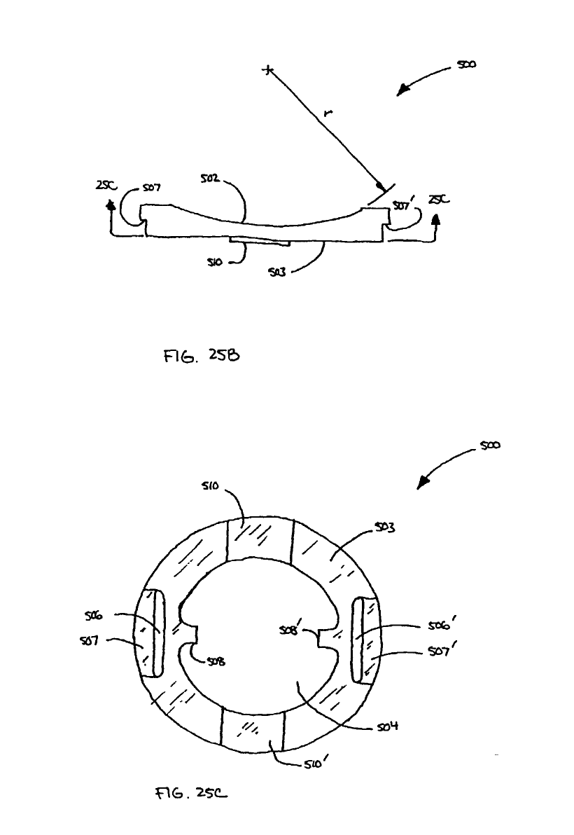

FIG. 25 is a perspective view of another embodiment of a mechanical stabilization device 499, illustrated in an exemplifying use environment. As shown, the stabilization device 499 comprises a first site selector 500 fastened to the forearm 462 and a second smaller site selector 500′ which is fastened to the finger 460. It is contemplated that each of the site selectors 500, 500′ is to be used in conjunction with a noninvasive monitor such as, but not necessarily limited to, the noninvasive system 10 as well as the apparatus taught in the above-mentioned U.S. Pat. No. 6,198,949. It is further contemplated that each of the site selectors 500, 500′ couples with, or otherwise operates in conjunction with a window of the noninvasive monitor and thus stabilizes the interface between the window and the subject's skin. Furthermore, it is contemplated that both the site selectors 500, 500′ may advantageously be used with the same noninvasive monitor. While the site selector 500 is sized for an optimal interface with the window of the noninvasive monitor, the site selector 500′ may be used with an adapter (not shown). Such an adapter facilitates effectively coupling the smaller site selector 500′ with the window of the noninvasive monitor. Additionally, it is contemplated that the site selectors 500, 500′ may be used in conjunction with the noninvasive monitor in accordance with the methodology taught in the above-mentioned U.S. Pat. No. 6,161,028.

A person of ordinary skill in the art will recognize that other techniques may advantageously be employed for placing the site selectors 500, 500′ in contact with the subject's skin. For example, in another embodiment the site selectors 500, 500′ may include an adhesive material which is adapted to attach the site selectors 500, 500′ to the skin. With this embodiment, each of the site selectors 500, 500′ includes a pressure sensitive adhesive surface which enables attaching the site selectors 500, 500′ to the subject's skin without using fastening straps.

FIG. 25A is a perspective view of one embodiment of the site selector 500. The illustrated embodiment of FIG. 25A is substantially similar to an apparatus described in Assignee's copending provisional application, entitled DEVICE FOR ISOLATING REGIONS OF LIVING TISSUE, Serial No. 60/311,521, filed Jul. 17, 2001, the entirety of which is hereby incorporated by reference. As shown in FIG. 25A, the site selector 500 is a generally flattened, rigid member comprising a contact surface 502, an interface surface 503, an aperture 504, openings 506, 506′, and protrusions 508, 508′. As illustrated in FIGS. 25B and 25C, the site selector 500 further comprises channels 507, 507′, and raised sections 510, 510′. The openings 506, 506′ and the channels 507, 507′ facilitate fastening the site selector 500 to the subject (see FIG. 25). The site selector 500 may be formed of injection-molded plastic or other similar material such that a noninvasive monitor, such as the noninvasive system 10 (FIG. 1) as well as the apparatus taught in the above-mentioned U.S. Pat. No. 6,198,949, may be coupled with the site selector 500 with minimal movement arising therebetween. Furthermore, it is contemplated that the material comprising the site selector 500 may be such that condensation formed thereon when the site selector 500 is exposed to cooler temperatures (below the dew point) is substantially minimized.

In an alternative embodiment, the site selector 500 may be made of a flexible, semi-compliant material which allows the site selector 500 to be bent such that it conforms to various regions of a patient's body. In one embodiment, the site selector 500 may be made of polyurethane. In another embodiment, the site selector 500 may be made of polypropylene. In still another embodiment, the site selector 500 may be made of silicone. Other embodiments may include other non-compliant or semi-compliant materials, or blends thereof, including but not limited to EVA (Ethylene-Vinyl-Acetate), PVC, PET, and NYLON. Those of ordinary skill in the art will recognize that the site selector 500 may advantageously be made of other non-compliant or semi-compliant, biocompatible materials.

The contact surface 502 presses against the subject's skin when the site selector 500 is strapped thereon or otherwise secured thereto. As can be seen most clearly in FIG. 25B, the contact surface 502 comprises a radius of curvature r which conforms to the topology of the location on the subject's body where the site selector 500 is intended to be used. In a preferred embodiment, wherein the site selector 500 is intended for use on the forearm 462, the contact surface 502 is curved and has a radius of curvature r of about 3.0 inches. It will be apparent to those skilled in the art that, depending upon where on the subject the site selector 500 is intended to be used, the contact surface 502 may advantageously be formed with other shapes or other radii of curvature r.

The interface surface 503 receives or otherwise engages with the above-mentioned noninvasive monitor. The protrusions 508, 508′ and the raised sections 510, 510′ respectively facilitate attaching and/or aligning the noninvasive monitor to the site selector 500. As will be appreciated by those of ordinary skill in the art, the configuration of the interface surface 503 (which, in the illustrated embodiment, includes a specific number, shapes, orientations, and characteristics of the protrusions 508, 508′ and the raised sections 510, 510′) is dependent upon the particular type of instrument with which the site selector 500 is intended to be used. On this basis, the number, shapes, orientations and characteristics of the protrusions 508, 508′ and the raised sections 510, 510′ (or the choice of structure used in place of or in addition to the protrusions 508, 508′ and the raised sections 510, 510′) may be substantially altered.

Referring to FIGS. 25A and 25C, the aperture 504 allows substantially unimpeded transmission of thermal spectra to and from the subject's skin through the site selector 500. The aperture 504 preferably has a substantially circular cross-section having a diameter of about 2.0 inches. It will be appreciated, however, that while in the embodiment of FIGS. 25A and 25C the aperture 510 has a circular cross-sectional shape, other cross-sectional shapes and sizes are contemplated, such as, by way of example, rectangular, circular, diamond, elliptical, and ovoid. It will further be appreciated that different cross-sectional shapes and sizes may advantageously be combined, thereby forming additional cross-sectional shapes.

Alternatively, the aperture 504 may comprise a substrate which serves as a thermal window. The substrate preferably is made of a material having a high thermal conductivity, such as polycrystalline float zone silicon or other similar material, such that the substrate is transparent to thermal spectra. In addition, the substrate may have a thickness sized such that thermal spectra are substantially unimpeded in passing through the substrate. It is contemplated that a suitable substrate which may be used with the site selector 500 of FIGS. 25A through 25C has a thickness of about 0.25 millimeters. It is further contemplated that the substrate has a cross-sectional shape and size such that the substrate is receivable by the aperture 504, thereby facilitating fastening of the substrate to the site selector 500. In one embodiment, the substrate may be permanently affixed within the aperture 504. In another embodiment, the substrate 504 may be removably inserted into the aperture 504. In the latter embodiment, the substrate may further comprise a disposable member which is attachable to and detachable from the site selector 500. It will be appreciated by those of ordinary skill in the art, however, that the substrate may be comprised of other materials, cross-sectional shapes and thicknesses.

As a further alternative, a heating element may be disposed upon the above-mentioned substrate such that the heating element contacts the skin when the site selector 500 is strapped to the subject (see FIG. 25). The heating element transfers heat to the skin of the subject, and thus gives rise to the heating component of the aforementioned intermittent heating and cooling of the subject's skin. As discussed with reference to FIGS. 24 and 24A, one embodiment of the heating element comprises an adhesion layer of gold deposited over an alloy layer which is applied to the substrate. The alloy layer comprises a material suitable for implementation of the heating element, such as 10/90 titanium/tungsten, titanium/platinum, nickel/chromium, or other similar alloy. The gold layer preferably has a thickness of 4000 Å, and the alloy layer preferably has a thickness ranging between 300 Å and 500 Å. Details regarding the manufacture and/or fabrication of the heating element are discussed herein with reference to FIG. 1 as well as in the above-mentioned U.S. Pat. No. 6,198,949.

Referring again to FIG. 25, the site selectors 500, 500′ are strapped to the forearm 462 and the finger 460, respectively, such that the contact surfaces 502, 502′ are pressed against the subject's skin, while the interface surfaces 503, 503′ face outward away from the skin. Pressure between the site selectors 500, 500′ and the subject's skin causes the perimeter of each aperture 504, 504′ of each of the site selectors 500, 500′ to “grip” the skin. This substantially minimizes relative motion between the skin and the site selectors 500, 500′. This gripping of the skin provides location stability whereby the site selectors 500, 500′ are prevented from sliding across the subject's skin when pushed or otherwise acted on by external forces, such as forces arising when the noninvasive monitor is attached and detached from the site selectors 500, 500′.

In operation, a noninvasive monitor, such as the noninvasive system 10 of FIG. 1 and the apparatus taught in U.S. Pat. No. 6,198,949, is placed in intimate contact with each of the interface surfaces 503, 503′ such that a window of each noninvasive monitor interfaces with the apertures 504, 504′ and is placed in thermal contact with the subject's skin. If, for some reason, either/or both of the noninvasive monitors must be temporarily removed from the subject's skin, such as to allow the subject mobility, the site selectors 500, 500′ may be left strapped to the forearm 462 and the finger 460 so as to maintain a consistent measurement site on the skin. When the noninvasive monitors are later reattached to the site selectors 500, 500′, the site selectors 500, 500′ will again place the windows of the noninvasive monitor in thermal contact with the same locations of skin as before. This substantially reduces measurement errors arising due to the otherwise variable nature of the contact between the noninvasive monitor and the subject's skin.

Although preferred embodiments and methods have been described in detail, certain variations and modifications thereof will be apparent to those skilled in the art, including embodiments and/or methods that do not provide all of the features and benefits described herein. Accordingly, the scope of the above-discussed embodiments and methods is not to be limited by the illustrations or the foregoing descriptions thereof, but rather solely by appended claims.

What is claimed is:

1. A method of determining a location on a subject's body whereat analyte measurements may be taken, based on the amount of elapsed time after the subject has eaten, said method comprising:
selecting an on-site location and an alternative site, said on-site location and said alternative site comprising distinct areas on said subject's body;
establishing a relationship between a restricted time period and said on-site location and between an unrestricted time period and said alternative site, said restricted time period commencing immediately after said subject eats, said unrestricted time period commencing immediately after said restricted time period terminates; and
determining whether said amount of elapsed time after said subject has eaten falls within during said restricted time period; and
restricting said subject to taking analyte measurements at said on-site location during the restricted time period.

2. The method of claim 1, further comprising permitting said subject to take analyte measurements at either said on-site location or said alternative site during said unrestricted time period.

3. The method of claim 2, wherein said alternative site is a forearm.

4. The method of claim 2, wherein said alternative site is a palm.

5. The method of claim 1, wherein said on-site location is a finger.

6. The method of claim 5, wherein said on-site location is a fingertip.

7. The method of claim 1, wherein said restricted time period is about 2.0 hours.

8. The method of claim 1, wherein said restricted time period lasts between about 0.5 hours and about 3 hours after said subject last ate.

9. The method of claim 1, wherein said restricted time period lasts between about 1.0 hours and about 2.0 hours after said subject last ate.

10. The method of claim 1, wherein said restricted time period lasts between about 1.5 hours and about 2.0 hours after said subject last ate.

11. A method of measuring analyte concentration within the living tissue of a subject at a measurement location on the body of said subject, said method comprising:
designating a restricted time period and an unrestricted time period, said restricted time period commencing immediately after said subject eats, said unrestricted time period commencing immediately after said restricted time period terminates;
selecting only an on-site measurement location during a restricted time period; and
selecting any of an on-site measurement location and an alternative-site measurement location during an unrestricted time period.

12. The method of claim 11, further comprising performing an analyte concentration measurement at the selected measurement site.

13. The method of claim 12, further comprising augmenting local circulation near the selected measurement site.

14. The method of claim 11, wherein performing an analyte concentration measurement comprises performing an invasive analyte concentration measurement.

15. The method of claim 14, wherein performing an invasive analyte concentration measurement comprises drawing a blood sample from said subject and determining analyte concentration in said blood sample.

16. The method of claim 11, wherein performing an analyte concentration measurement comprises performing a noninvasive analyte concentration measurement.

17. The method of claim 16, wherein performing a noninvasive measurement comprises using an optical measurement system.

18. The method of claim 17, wherein said optical measurement system comprises a thermal gradient spectrometer which detects infrared energy emitted and/or reflected by said subject's tissue to determine said analyte concentration based on the amount of infrared energy absorbed by the analyte.

19. The method of claim 11, wherein performing an analyte concentration measurement comprises performing an invasive analyte concentration measurement only at one of said on-site measurement location and said alternative-site measurement location, and performing a noninvasive analyte concentration measurement only at the other of said on-site measurement location and said alternative-site measurement location.

20. The method of claim 11, wherein said alternative-site measurement location is a forearm.

21. The method of claim 11, wherein said on-site measurement location is a finger.

22. The method of claim 21, wherein said on-site location is a fingertip.

23. The method of claim 21, wherein said alternative site is a palm.

24. The method of claim 11, wherein said restricted time period is about 2.0 hours.

25. The method of claim 11, wherein said restricted time period lasts between about 0.5 hours and about 3 hours after said subject last ate.

26. The method of claim 11, wherein said restricted time period lasts between about 1.0 hours and about 2.0 hours after said subject last ate.

27. The method of claim 11, wherein said restricted time period lasts between about 1.5 hours and about 2.0 hours after said subject last ate.

28. A mechanical stabilization device for immobilizing a finger and/or a hand for exposure to a blood constituent monitor, said device comprising:
a base comprising an elbow channel and a forearm channel for respectively stabilizing an elbow and a forearm of an arm such that relative movement between said arm and said base is substantially minimized, said forearm channel including a primary window configured for thermal contact with said forearm; and
a finger restraint comprising a finger hole which includes a secondary window configured for thermal contact with said finger.

29. The device of claim 28, wherein said primary window is configured to interface with said blood constituent monitor, said primary window facilitating capturing of analyte concentration data within tissue of said forearm.

30. The device of claim 28, wherein said secondary window is configured to interface with said blood constituent monitor, said secondary window facilitating capturing of analyte concentration data within tissue of said finger.

31. The device of claim 28, wherein said elbow channel and said forearm channel respectively conform to the anatomical shapes of said elbow and said forearm of said arm.

32. The device of claim 28, wherein said finger restraint conforms to the anatomical shape of said finger.

33. The device of claim 28, wherein said base further comprises a pair of forearm restraining holes and a pair of elbow restraining holes, said forearm restraining holes and said elbow restraining holes facilitating stabilizing said arm within said base.

34. The device of claim 28, wherein said finger restraint is movable distally and proximally relative said forearm channel to accommodate various forearms and fingers having different lengths.

35. The device of claim 28, wherein said finger hole has a diameter which may be increased and decreased so as to stabilize a variety of fingers having different sizes.

36. A method for stabilizing an arm and a finger of a subject for determination of analyte concentration within said subject's tissue, said method comprising:
providing a mechanical stabilization device comprising a base and a finger restraint, said base comprising an elbow channel and a forearm channel for stabilizing said arm, said forearm channel including a primary window configured for thermal contact with said forearm, said finger restraint comprising a finger hole which includes a secondary window;
inserting said finger into said finger hole while said forearm is laid onto the forearm channel and said elbow is placed within the elbow channel;
securing said forearm within said forearm channel and securing said elbow within said elbow channel, such that said forearm is placed into thermal contact with said primary window;
tightening said finger hole around said finger such that said finger is placed into thermal contact with said second window; and
performing said determination of analyte concentration within said subject's tissue.

37. The method of claim 36, wherein said finger hole has an adjustable diameter which can be increased and decreased so as to tighten around and release said finger.

38. The method of claim 36, wherein said securing said forearm further comprises passing a forearm fastening strap over said forearm and through a pair of forearm fastening holes within said base, said fastening strap tightened to prevent relative movement between said forearm and said forearm channel.

39. The method of claim 36, wherein said securing said elbow further comprises passing an elbow fastening strap over a proximal portion of said forearm and through a pair of elbow fastening holes within said base, said fastening strap tightened to prevent relative movement between said elbow and said elbow channel.

40. A mechanical stabilization device for use with a monitor for determining analyte concentration within tissue of a subject, said device comprising:
a first site selector forming a thermal interface between a window of said monitor and an on-site location of said tissue; and
a second site selector forming a thermal interface between said window of said monitor and an alternate site on said tissue, said on-site location and said alternate site comprising two distinct locations on said tissue of said subject.

41. The device of claim 40, wherein said first site selector is smaller than said second site selector.

42. The device of claim 41, further comprising an adaptive member which facilitates coupling said first site selector with said monitor.

43. The device of claim 40, wherein said alternate site is a forearm.

44. The device of claim 40, wherein said on-site location is a finger.

45. The device of claim 40, wherein said first and second site selectors each comprises a generally flat member having an aperture which allows thermal spectra to pass therethrough.

46. The device of claim 45, wherein said first and second site selectors each interfaces with a window of said monitor.

47. The device of claim 40, wherein said first and second site selectors are each made of a flexible, semi-compliant material which allows said first and second site selectors to bend thereby conforming to various location on said subject.

48. The device of claim 40, wherein said first and second site selectors each comprises a window having a heating element disposed thereon, each of said windows comprising a material of high thermal conductivity so as to permit thermal spectra to pass therethrough.

49. The device of claim 48, wherein said first and second site selectors are each electrically connected to an external power supply.

50. The device of claim 48, wherein a power cable places said first site selector in electrical communication with said second site selector whereby said first site selector receives electric power when said second site selector is connected to said external power supply.

51. The device of claim 40, wherein fastening straps are used to attach said first and second site selectors to said tissue of said subject.
